(19) United States
(12) Patent Application Publication
(10) Pub. No.: US 2024/0191217 A1
(43) Pub. Date: Jun. 13, 2024

(54) CAS10-BASED ASSAY FOR NUCLEIC ACID DETECTION

(71) Applicant: UNIVERSITY COURT OF THE UNIVERSITY OF ST ANDREWS, St Andrews, Fife (GB)

(72) Inventors: Sabine Gruschow, St. Andrews (GB); Malcolm F. White, St. Andrews (GB)

(21) Appl. No.: 18/287,325

(22) PCT Filed: Apr. 14, 2022
(86) PCT No.: PCT/GB2022/050960

§ 371 Date: Oct. 18, 2023

(30) Foreign Application Priority Data
Apr. 20, 2021 (GB) 2105620.5
Sep. 10, 2021 (GB) 2112941.6

(51) Int. Cl. C12N 9/22; C12Q 1/682; C12Q 1/70

(52) U.S. Cl. CPC C12N 9/22; C12Q 1/682; C12Q 1/701

(57) ABSTRACT

Detection system which comprise a CRISPR-associated effector protein programmed with a molecule binding (by complementary base pairing) a target nucleic acid. On binding of an RNA, Cas10 can generate cyclic oligoadenylate (COA) which in turn activates a nuclease, e.g. NucC. The nuclease can, via a reporter system, generate a readily detectable fluorescent/coloured signal. Preferred embodiments employ the Cas10 and NucC from Vibrio metoccus.

### BACKGROUND

Infectious agents such as the SARS-COV-2 virus, causative agent of Covid-19, constitute a public health challenge. A key weapon in our efforts to combat this challenge lies in technology to detect the virus in samples from patients. The ideal test will be specific (low false positives), sensitive (low false negatives), fast (high throughput), scalable and cost effective.

The gold standard assays for detection of viral RNA involve the conversion of the RNA into DNA by reverse transcription (RT), then amplification of the DNA using the polymerase chain reaction (PCR). This “RT-PCR” methodology requires complex thermocycling apparatus for the amplification, and detects a fluorescent product that is formed when the viral RNA is present in samples. The process typically takes 1-2 hours to complete.

Alternative techniques use isothermal (one temperature) amplification to avoid the need for PCR. They still typically require conversion of viral RNA into DNA, which is then amplified. Recent developments have included the use of the CRISPR effector Cas12, commercialised as DETECTR, which detects viral DNA and generates a fluorescent signal (4). A second approach, named SHERLOCK, uses the CRISPR effector Cas13, which can be coupled with a ribonuclease called Csm6, to detect viral RNA and generate a fluorescent signal (5).

The present disclosure provides a new CRISPR-based approach to the detection of the infectious agents in samples.

### SUMMARY

The disclosure provides a system comprising a CRISPR-associated effector protein programmed with a molecule binding (by complementary base pairing) a target nucleic acid. This binding event can be coupled to nucleases that, via a reporter system, can generate a readily detectable fluorescent/coloured signal.

Type III CRISPR systems comprise the Cas10 enzymatic subunit. On detection of a specific nucleic acid (RNA), Cas10 can generate cyclic oligoadenylate (cOA). The system of this disclosure exploits this effect in the detection of target nucleic acid sequences.

More specifically, the Cas 10 protein is activated to generate cOA by binding between a molecule which binds a target nucleic acid via complementary base-pairing. The system can be tailored to the detection of any target nucleic acid sequence by altering the specificity of the molecule which binds the target nucleic acid. An advantage of the system is that it generates large quantities of cOA for every single nucleic acid (RNA) molecule detected

Accordingly, this disclosure provides systems and methods for the detection of nucleic acid sequences. The systems and methods described herein can be applied to the detection of nucleic acid, in particular RNA, in a sample and as a consequence, find application in the diagnosis and/or detection of diseases, conditions and/or pathogens.

One particular application of the systems and methods of this disclosure is in the diagnosis of viral pathogens and the diseases and/or conditions caused and/or contributed to thereby. The systems and methods may be applied to the detection of RNA from a wide range of viral pathogens, including, for example, SARS-COV-2, SARS-COV-1 or MERS-COV and in the diagnosis of the diseases and/or conditions they cause (e.g. Covid-19, SARS and MERS).

In one teaching, there is provided a system for the detection of a target nucleic acid, said system comprising
a CRISPR associated protein 10 (Cas10);
a molecule, capable of binding the target nucleic acid or a molecule which can be processed into a molecule which binds the target nucleic acid;
a nuclease; and
a reporter system.

As is described in more detail below, the part of the system for binding the target nucleic acid is provided in the form of a molecule which inherently and/or directly binds the target nucleic acid (or at least some target site within the target nucleic acid) or, as a molecule which is (or can be) processed to yield a molecule which has the ability to bind the target nucleic acid or at least a target site within the same. For convenience, this molecule shall be referred to herein after as either “a molecule or processed form thereof capable of binding the target nucleic acid” and/or the “molecule/processed molecule”. For the avoidance of doubt, the molecule/processed molecule is (i) capable of binding the target nucleic acid and (ii) upon binding the target nucleic acid, activates the Cas 10 component of the system to generate a quantity of cOA.

The disclosure further provides a use of a system described herein for the detection of a target nucleic acid. By way of example, a system described herein may be used to probe a sample for the presence of a target nucleic acid sequence and/or for the diagnosis of a disease or condition.

The disclosure provides the use of a system described herein in a method for the detection of a target nucleic acid in a sample and/or in a method of diagnosing a disease or condition.

Also disclosed is a method of detecting a target nucleic acid in a sample, said method comprising contacting a sample with a system described herein, wherein detection of a signal from the reporter system, indicates that the sample contains the target nucleic acid. The method may be an in vitro method.

It should be noted that the terms “comprise”, “comprising” and/or “comprises” is/are used to denote that aspects and embodiments of this invention “comprise” a particular feature or features. It should be understood that this/these terms may also encompass aspects and/or embodiments which “consist essentially of” or “consist of” the relevant feature or features.

Without wishing to be bound by theory, when a system of this disclosure is brought into contact with a sample containing the target nucleic acid, binding between the molecule/processed molecule and the target nucleic acid activates the Cas 10 component. Activation of Cas10 in turn leads to activation of the nuclease. The activated nuclease then interacts with the reporter element in order to release a detectable signal. Detection of that signal will indicate (to a user of the system) that the sample contains the target nucleic acid.

In contrast (and again without being bound by theory), where the sample does not contain the target nucleic acid, upon contact with a system of this disclosure, there will be no binding event between the molecule/processed molecule and the target nucleic acid. Likewise, there will be no associated Cas10 activation and the nuclease element will remain ‘inert’ and not able to interact with the reporter. There will be no detectable signal and the lack of that signal will indicate that the sample does not contain the target nucleic acid.

A particular advantage of the systems and methods described herein is the limit of detection (LOD). The disclosed systems and methods enable the detection of very low amounts of target nucleic acid (for example, very low amounts of SARS-COV-2 nucleic acid). For example, a system or method of this disclosure may enable a LOD of about <1 fM to 90 fM; for example 1 fM, 2 fM, 3 fM, 4 fM, 5 fM, 6 fM, 7 fM, 8 fM, 9 fM, 10 fM, 20 fM, 30 fM, 40 fM, 50 fM, 60 fM, 70 fM, 80 fM or 90 fM. This level of detection is achievable even without an extrinsic amplification step. It should be noted that in the case of a target viral nucleic acid (for example a target SARS-COV-2 nucleic acid) a LOD of <1 fM may equate to about 1×104 molecules of the virus.

The target nucleic acid may comprise RNA.

Accordingly, the molecule/processed molecule may comprise a molecule capable of binding a target RNA or may be processed to yield a molecule capable of binding a target RNA. In this way, the various uses and methods described herein may be for the detection of RNA in a sample.

The target nucleic acid may be a human or animal nucleic acid sequence, including human/animal RNA sequences. For examples, the nucleic acid may be a gene transcript.

The nucleic acid may comprise an oncogene transcript and/or a mutated RNA arising from a mutated gene.

The target nucleic acid may comprise nucleic acid (including transcripts) from a pathogen. Nucleic acid of this type may be referred to as ‘pathogen associated’ or ‘pathogen derived’ nucleic acid.

The target nucleic acid may comprise nucleic acid, including RNA, from a microorganism (microbial (including bacterial) RNA/nucleic acid).

The target nucleic acid may comprise nucleic acid, including RNA, from a virus (viral RNA/nucleic acid).

Thus, target nucleic acid may comprise, consist essentially or consist of a pathogen associated/derived RNA and/or a viral RNA.

The target nucleic acid may comprise nucleic acid, including RNA, from, for example, a Coronavirus, SARS-COV-1, SARS-COV-2 or MERS-COV.

Where the target nucleic acid is a viral nucleic acid/viral RNA, the nucleic acid may be derived from one or more specific genes. A viral derived nucleic acid may include a transcript derived from any part of the genome.

In the case of SARS-COV-2, the target nucleic acid may be derived from any part or parts of the single single-stranded RNA genome, including those sections that encode the spike protein (S) and/or membrane protein (M) and/or envelope protein (E) and/or Nucleoprotein (N).

An exemplary target sequence may comprise any part of the SARS-COV-2 nucleoprotein sequence. By way of example, exemplary target sequences may comprise nucleotide 57, 78, 209, 230, 633, 719, 782, 909, 980 or 1169 of the sequence encoding the nucleoprotein. Each target sequence may, for example, start at one of the noted nucleotides and comprise 10-50 nucleotides of the nucleoprotein encoding sequence. The precise number of nucleotides of the target sequence may not be crucial, but a target sequence of about 15, 20, 25, 26, 27, 28, 29, 30, 31, 32, 33, 34, 35, 36, 37, 38, 39, 40 or 45 nucleotides may be preferred. A SARS-COV-2 target sequence may comprise (consist of or consist essentially of) any of SEQ ID NOS: 1 or 2 shown below:

SEQ ID NO: 1
AAGGCGUUCCAAUUAACACCAAUAGCAGUCCA
SEQ ID NO: 2
GAUGGUAUUUCUACUACCUAGGAACUGGGCCA

A system of this disclosure may comprise a molecule, capable of binding the target nucleic acid or a molecule which can be processed into a molecule which binds the target nucleic acid (for example as explained in more detail below, a guide RNA) which molecule comprises a sequence designed to match a part (or portion) of the target sequence. By way of example, where the target is SARS-COV-2, the target nucleic acid binding part of the disclosed system may comprise a sequence designed to match a part (or portion) of the nucleoprotein encoding sequence of the SARS-COV-2 genome, for example a sequence which binds, matches or is complementary to, all or part of the sequences given as SEQ ID NOS: 1 and 2 above.

In view of the above, the disclosure provides a method of detecting SARS-COV-2 or a nucleic acid thereof, in a sample, said method comprising probing the sample for the presence of SEQ ID NO: 1 or 2.

The disclosure further provides the use of a probe or nucleic acid which binds or is complementary to SEQ ID NO: 1 or 2, in a method of detecting a SARS-COV-2 nucleic acid in a sample.

A sample (for example a biological sample, saliva, blood (or a fraction thereof) a tissue scraping, biopsy, cell, tissue/organ secretion or wash) which is found to contain either or both of SEQ ID NO: 1 may comprise SARS-COV-2 nucleic acid. A sample may be provided by a subject with a SARS-COV-2 infection, a subject recovering or convalescing from a SARS-COV-2 infection, a subject suspected of having a SARS-COV-2 infection and/or a subject susceptible or predisposed to a SARS-COV-2 infection.

A method of detecting SARS-COV-2 nucleic acid may exploit a Cas10-based system, such as, for example, the system described herein. A method for detecting a SARS-COV-2 nucleic acid may comprise a VmeCmr system of this disclosure. The guide RNA component of a useful VmeCmr system may comprise, for example, a sequence which represents a match (which is complementary to or which binds) all or part of the target sequences represented by SEQ ID NOS: 1 and 2 above.

Without wishing to be bound by theory, it is suggested that an advantage of any method based around the detection of the target sequences given as SEQ ID NOS 1 or 2 herein, is that of a highly specific assay with improved sensitivity. For example, the limit of detection (of a target nucleic acid, for example SARS-COV-2 nucleic acid) of an assay of this type is in the region of <1 fM, or about 10 fM to about 80 fM. For example the LOD (of a target nucleic acid, for example SARS-COV-2 nucleic acid) may be in the region of 1 fM, 2 fM, 3 fM, 4 fM, 5 fM, 6 fM, 7 fM, 8 fM, 9 fM, 10 fM, 20 fM, 30 fM, 40 fM, 50 fM, 60 fM, 70 fM, 80 fM or 90 fM. This level of detection is achievable even without an extrinsic amplification step.

The disclosure further provides a kit for the detection of SARS-COV-2 nucleic acid in a sample, the kit comprising a probe for the target sequence(s) SEQ ID NO: 1 and/or 2. The kit may comprise (or further comprise) a system as disclosed herein. The kit may optionally further comprise buffers, diluents, receptacles and/or instructions for use.

The methods of this disclosure and applications of the systems described herein should not be construed as being limited to the detection of a specific RNA from any specific pathogen—almost any nucleic acid/RNA sequence (natural or synthetic), including any pathogen associated/derived nucleic acid/RNA can be detected using the methods and/or systems described herein.

Moreover, a system, use or method described herein may be applied to the detection of one, two, three or more (i.e. a plurality of) different target nucleic acid/RNA sequences in a sample.

Without wishing to be bound by theory, human or animal subjects that are, or that have been, infected with a particular pathogen and/or that are suffering from or have suffered from a disease or condition caused by a particular pathogen may yield samples comprising amounts of nucleic acid associated with that pathogen. As such, the systems, methods and uses described herein (which systems methods and uses can be applied to the detection of specific nucleic acids in samples) may be used to diagnose or detect diseases and/or infections associated with certain pathogens.

Where a system, use or method of this disclosure is to be applied to the diagnosis of a disease, the target nucleic acid may be derived from a pathogen associated with the disease and/or condition to be diagnosed. It should be understood that detection of the target nucleic acid in a sample, will indicate that the sample has been obtained from or provided by a subject infected with (or who has been infected with) the pathogen associated with the target nucleic acid.

Accordingly, where the disclosed system, use or method is to be applied to the detection of a viral pathogen, viral disease and/or viral condition, the target nucleic acid may be derived from, or associated with, the relevant virus—i.e. the virus causing or contributing to the disease and/or condition to be diagnosed. In this way, detection of the viral associated target nucleic acid in a sample will indicate that that sample has been provided by, or obtained from, a subject infected with (or who has been infected with), the virus/viral pathogen and/or a subject suffering from (or who has been suffering from), a diseases and/or condition caused by the viral pathogen.

By way of a non-limiting example, where the disclosed system, use or method is to be applied to the detection of a Coronavirus (for example SARS-COV-1, MERS-COV or SARS-CoV-2) infection and/or the diagnosis of a disease or condition caused or contributed to by the same (for example Covid-19, SARS or MERS), the target nucleic acid may be derived from or associated with the relevant Coronavirus pathogen. One of skill will appreciate that detection of a Coronavirus associated target nucleic acid in a sample, indicates that the sample has been obtained from or provided by a subject who has, or has had, a Coronavirus infection.

It should be noted that a key advantage of the systems, methods and uses described herein is that they (or their use) can achieve a sensitivity for a particular target nucleic acid that is far more sensitive that prior art systems, methods and uses. As a consequence, the systems described herein and the associated methods and uses provide the prospect of the rapid, sensitive and accurate detection of any desired target nucleic acid/target RNA without the need for any additional amplification steps.

It should be noted that target nucleic acids might be detected in a wide variety of different sample types. Indeed the specific sample type may vary depending on the nucleic acid that is to be detected. For example, the sample may comprise any human or animal sample. Moreover, in order to detect some types of pathogen (for example viral) associated/derived nucleic acid, blood samples may be used whereas the detection of other pathogen (viral) associated/derived nucleic acids may require the use of samples comprising saliva, mucus, tissue, cells and the like. The choice of sample will depend on the pathology of the pathogen; nevertheless, any sample that might contain nucleic acid (in particular the target nucleic acid) can be subjected to a method of this disclosure.

A sample may be subject to a nucleic acid extraction process to yield nucleic acid, for example RNA, for use in a method of this disclosure and/or for contact with a system described herein.

One of skill will know of the various techniques that can be used to extract nucleic acid, including RNA, from a sample and all of those techniques can be used here.

Suitable techniques for the extraction of nucleic acids from biological samples may include organic extraction methods (using, for example, phenol/chloroform), inorganic extraction methods (salting out) and/or solid phase extraction methods (using some form of solid matrix). For example spin columns comprising a solid phase matrix designed for the purpose of nucleic acid (RNA) extraction, can be used.

A nucleic acid extraction protocol may involve a step in which the nucleic acid is isolated and further steps in which the nucleic acid is purified and/or collected for use.

A sample and/or any nucleic acid extracted or purified therefrom may be further subjected to some form of amplification protocol—the aim being to increase the amount of nucleic acid present and which is subjected to the methods described herein. The reader will appreciate that anything which increases the amount of nucleic acid to be subject to any of the uses and/or methods described herein, may positively impact on the sensitivity of the method/use and may permit detection of nucleic acid in samples which contain low/substantially undetectable levels of nucleic acid. Any amplification technique may be used including, for example PCR-based techniques, isothermal nucleic acid amplification (including LAMP and the like)

A nucleic acid to be subjected to a use or method of this disclosure may be converted from DNA to RNA. Such conversion may take place prior to, before and/or after any purification and/or amplification protocols.

The extracted, amplified and/or purified nucleic acid, or an amount thereof, may then be subjected to any of the methods described herein.

In one teaching, the disclosure provides a method of detecting a target nucleic acid in a sample, said method comprising:
providing a sample and contacting the sample with a system described herein.

In any method of detecting a target nucleic acid in a sample, the detection of a signal from the reporter system indicates that the sample contains the test nucleic acid.

A method of detecting a target nucleic acid in a sample may further comprise contacting a disclosed system with the sample under conditions which permit binding between the molecule/processed molecule and any target nucleic acid present in the sample. As stated (and without being bound by theory), binding between the molecule/processed molecule and the target nucleic acid ‘activates’ the Cas10 component of the system.

To facilitate activation of the nuclease (by activated Cas10), the system may further comprise ATP. Activated Cas10 will, in the presence of ATP, generate cyclic oligoadenylate (cOA) molecules. Specifically, in the presence of ATP, Cas10 will generate cyclic tri-adenylate (cA₃). The generated cyclic tri-adenylate(cA₃) will activate the nuclease component of the system. The activated nuclease then acts on the reporter system to release a detectable signal.

The system may comprise 1 μM, 10 μM, 50 μM, 100 μM, 150 μM, 200 μM, 250 μM, 300 μM, 350 μM, 400 μM, 450 μM, 500 μM, 550 μM, 600 μM, 650 μM or 700 μM ATP. The precise amount of concentration of ATP may vary and it should be appreciated that the amount of ATP should be sufficient to enable the generation of cyclic oligoadenylate (cOA) molecules by the Cas10 component (when activated by a binding event between the molecule/processed molecule (via base-pairing) and the target nucleic acid itself)).

In a method of detecting a target nucleic acid in a sample, the method may comprise a step in which ATP is added. As stated, but without being bound by theory, the addition of ATP permits activated Cas10 (activated by binding between the molecule/processed molecule and the target nucleic acid). The ATP may be added prior to during and/or after any step in which the system of this disclosure is brought into contact with the sample.

In one teaching, a method of detecting a target nucleic acid in a sample, may comprise:
providing a sample and contacting the sample with a system described herein;
incubating the sample and system for a time and under conditions which permit binding between any target nucleic acid present in the sample and the molecule/processed molecule.

Before, during or after the providing step, a method of detecting a target nucleic acid in a sample may further include a step comprising the addition of ATP.

Alternatively, before, during or after the incubating step, a method of detecting a target nucleic acid in a sample may further include a step comprising the addition of ATP.

As noted above, ATP may be added at any of the abovementioned amounts/concentrations.

In any method of detecting a target nucleic acid in a sample, the detection of a signal from the reporter system indicates that the sample contains the test nucleic acid. Again, and without wishing to be bound by theory, binding between target nucleic acid in the sample and the molecule/processed molecule activates the Cas10 component of the system and activated Cas10 will generate a large quantity of cyclic tri-adenylate (cA3), from ATP. The generated cA₃ activates the nuclease component and this converts the reporter system into a detectable signal.

It should be noted that the conversion of ATP to cyclic tri-adenylate (cA₃) by activated Cas10 provides a means of signal amplification. In contrast to prior art systems which might use or exploit other enzymes, Cas10 generates a large quantity of cyclic tri-adenylate (cA₃). Thus, from a small amount of target nucleic acid, the system can generate a relatively large and easily detectable signal. Without wishing to be bound by theory, for every single RNA molecule detected, up to about 3000 cOA molecules (i.e. (cA₃)) may be generated by a system described herein; this represents a significant amplification step.

The specificity of a system of this disclosure is determined by the specificity of the molecule/processed molecule which binds to the target nucleic acid. In other words, the design of the molecule/processed molecule, programmes the system of this disclosure—making it capable of detecting a specific and/or predetermined target nucleic acid (RNA) sequence.

By changing features of the nucleic acid binding molecule (or the nucleic acid-binding processed form), it is possible to devise systems capable of detecting the presence of one or more specific target nucleic acids.

The molecule/processed molecule not only functions to bind to the target nucleic acid, but upon binding (which may occur via complementary base-pairing) it activates Cas 10 to convert ATP into cOA (specifically cA₃).

This component of the disclosed system may itself comprise a nucleic acid which (a) binds to a target nucleic acid sequence (or a site therein) or (b) is processed to a nucleic acid molecule which binds to a target nucleic acid sequence (or a site therein). The nucleic acid of the molecule/processed molecule may comprise RNA.

The molecule may be processed by Cas6 to yield a processed molecule which is capable of binding a target nucleic acid, for example a target RNA, or a target site therein. Accordingly, the processed molecule may be a Cas6 processed molecule.

A molecule/processed molecule may comprise a nucleic acid sequence which is complementary to all or part of the target nucleic acid sequence. Where the target nucleic acid is an RNA, the molecule/processed molecule may comprise a (RNA) sequence which is complementary to all or part of that target RNA sequence. One of skill will appreciate that binding between two complementary nucleic acid sequences (e.g. two complementary RNA sequences) will occur via complementary base pairing.

Where the molecule/processed molecule comprises a nucleic acid sequence, the sequence or part of the sequence, of that nucleic acid will determine the target nucleic acid to which the molecule binds. In this case, the target nucleic acid will have a sequence at least partially complementary to a sequence of the molecule which binds it or which is processed to bind it. The molecule/processed molecule may comprise one or more of these sequences which may otherwise be known as ‘spacer’ sequences. The sequence of each of the various spacer sequences may be at least partially complementary to sequences present in the target nucleic acid.

The molecule/processed molecule may comprise a plurality (for example) two, three, four, five, six or more of these spacer sequences. Each spacer sequence may comprise a sequence at least partially complementary to a specific region of the target nucleic acid.

The design of these spacer sequences may vary and the sequence of any of the spacer sequences need not be 100% complementary to the corresponding sequence in the target nucleic acid (in other words, they need only be partially complementary). An advantage of the CRISPR type III system and/or the Cas10 element derived therefrom, is that it is tolerant of extensive mis-paring between the abovementioned spacer sequences and the target nucleic acid (RNA) sequence. This advantage extends into the current system where there may be some degree of mis-match between any of the spacer sequences of the molecule/processed molecule and the corresponding sequences of the target nucleic acid. These mis-matches do not necessarily affect the performance of the system which remains able to accurately and sensitively detect the presence of a target nucleic acid in a sample.

A further advantage associated with any of the described systems, uses and/or methods is that the systems, methods and/or uses can discriminate between nucleic acid variants with as little as a single nucleotide polymorphism (SNP). This makes the technology described in this disclosure particularly suitable for the detection of pathogen associated nucleic acids which, when derived from different strains and species, can be highly variable with SNP differences. This applies in particular to the detection of SARS-COV-2 associated nucleic acid, which between strains and variants, may differ by the presence of single mutations. In one teaching, the molecule/processed molecule, capable of binding the target nucleic acid, may comprise one or more spacer sequences which are designed to bind to any form or variant of the target nucleic acid. As stated, binding between the molecule/processed molecule and the target nucleic acid activates the Cas10 component of the system.

A spacer sequence may be at least 65%, 70%, 75%, 80%, 85%, 90%, 91%, 92%, 93%, 94%, 95%, 96%, 97%, 98%, 99% or 100% complementary to a sequence within the target nucleic acid. In other words, at least 65%, 70%, 75%, 80%, 85%, 90%, 91%, 92%, 93%, 94%, 95%, 96%, 97%, 98%, 99% or 100% of the bases of a spacer sequence may be complementary to the corresponding sequence in the target nucleic acid.

By way of non-limiting example, where the target nucleic acid is a SARS-COV-2 RNA sequence, the molecule/processed molecule may comprise one or more spacer sequences comprising sequences which are at least partially complementary to one or more SARS-COV-2 sequences. A molecule/processed molecule capable of binding a SARS-COV-2 nucleic acid may comprise, for example, five complementary sequence, with each sequence comprising nucleobases complementary to sequences within the SARS-COV-2 N gene.

A molecule/processed molecule may comprise a (synthetic) CRISPR RNA (crRNA). The CRISPR RNA may comprise one or more sequences complementary to at least part of the sequence of the target nucleic acid.

A molecule/processed molecule may comprise a guide RNA. A (or the) guide RNA may comprise a sequence complementary to at least part of the sequence of the target nucleic acid—e.g. to a target sequence site within the target nucleic acid. A guide RNA may be derived from a molecule comprising two elements: the CRISPR repeat and the spacer. The repeat sequence is cleaved by Cas6 to make (or yield) a guide RNA. The guide RNA would then bind to the target nucleic acid (via complementary base pairing). Accordingly, a system of this disclosure may comprise a guide RNA for binding a target nucleic acid and/or a target site within the target nucleic acid. As stated, the system may comprise a molecule which comprises one or more guide RNA(s), each guide RNA having affinity and/or binding specificity for a particular target nucleic acid and/or a target site within the same. The molecule may be processed, by for example Cas6, to release the one or more guide RNA(s) which are then loaded into the Cas10 part of the system. Binding between the one or more guide RNA(s) and the target nucleic acid (or the target site within the same) will activate the Cas10 and this in turn generates cA3 which activates the nuclease component of the system.

A guide RNA may comprise a number of (for example 8)5′ nucleotides derived from the repeat (this is sometimes called the 5′-handle) and a number of nucleotides (for example, approximately 33 nucleotides) that are complementary to the target RNA. The sequence of the 5′-handle may not base pair with the target nucleic acid (RNA).

The molecule/processed molecule may comprise a CRISPR locus. The CRISPR locus may comprise one or more sequences complementary to at least part of the sequence of the target nucleic acid.

The molecule/processed molecule may comprise a promoter element.

The molecule/processed molecule may further comprise one or more repeat sequences. These repeat sequences may flank any sequences that are designed to be complementary to a sequence of a target nucleic acid (i.e. they may flank the spacer sequences). Any, for example Cas6-based, processing to which the molecule is subjected (to yield a processed molecule) may remove repeat sequences and present guide RNAs (derived from, or corresponding to, the spacer sequences) which can be ‘loaded’ into the Cas10 component and made available for binding to the target nucleic acid sequence.

The molecule/processed molecule may comprise one or more of (i) a promoter sequence, (ii) one or more repeat sequences and/or (iii) one or more spacer sequences, wherein the one or more repeat sequences may flank one, more or all of the spacer sequences and spacer sequences comprise a sequence which is complementary to a sequence of the target nucleic acid.

It will be understood that the sequence of the target nucleic acid will, to some extent, dictate the design of the spacer sequence/guide RNA for use in a system of this disclosure (as the molecule/processed molecule which binds to the target nucleic acid).

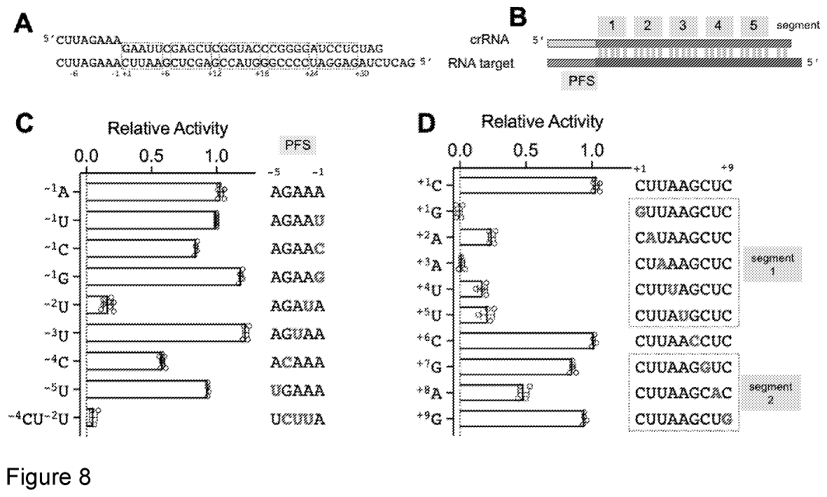

FIG. 8 refers to some design principles, which can be applied to the design of exemplar guide RNA molecules.

For example, in order to generate a suitable guide RNA (or a molecule yielding a suitable guide RNA after (e.g. Cas6) processing), one might first select a target site in the target nucleic acid. The guide RNA would then be designed to bind that target site within the target nucleic acid.

A target site may be selected according to the following rules:
1. SNPs in positions +1 to +5, and particularly at +1 and +3, may effect Cas10 activation. By careful selection of a target sequence, target nucleic acid (RNA) sequences differing by a single point mutation can be discriminated. In one teaching a target sequence (or target site within the same) may comprise SNPs at position +1 or +3—this would ensure maximum discrimination potential.
2. The sequence of the protospacer flanking sequence (PFS) may also be selected maximise Cas10 activation. For example, a sequence with a Guanine at position −1 may improve Cas10 activation. One may also avoid a base pairing event at position −2, as this may adversely affect Cas10 binding.

The reader will understand that one or more of these target sequence selection rules may be used to modulate Cas10 activation in a system or method of this invention. By way of example, these rules can be used to either positively or negatively affect the sensitivity of any of the methods described herein.

The system may further comprise a Cas6 element.

Additionally or alternatively, the system may comprise one or more self-cleaving ribozymes.

Accordingly, a system of this disclosure may comprise
a CRISPR associated protein 10 (Cas10);
a molecule or processed form thereof, capable of binding the target nucleic acid;
a nuclease;
a reporter system;
optionally ATP; and
optionally Cas6 and/or self-cleaving ribozymes; wherein the molecule/processed molecule binds the target nucleic acid by complementary base pairing. Furthermore, upon binding between the molecule/processed molecule and the target nucleic acid, Cas10 is activated and generates cA₃ (by, for example, converting any ATP added to the system to CA₃).

The CRISPR Cas10 component may be derived from a type III CRISPR complex.

By way of non-limiting example, Cas10 proteins for use may be found, for example, within the type III CRISPR systems of microorganisms classified as belonging to the Gamma Proteobacteria, the Firmicutes and the Bacteroidetes.

Useful Cas10 components may be those that generate cA₃ in the presence of ATP. One of skill will appreciate any given Cas10 may be tested for an ability to generate cA₃ by creating a system according to this disclosure in which the Cas10 component is a ‘test’ component with an undetermined ability to generate cA₃ from ATP. The system may further comprise a guide RNA or a molecule processed in a way to yield a guide RNA, which is capable of binding a predetermined nucleic acid sequence. The system may then be brought into contact with the relevant target nucleic acid and binding between that nucleic acid and the guide RNA part of the system will ‘activate’ the Cas10 component. Thereafter, if the test Cas10 component is able to generate cA₃ from ATP, cA₃ will be detected. If no cA₃ is detected or another cOA (for example cA₄ or the like), the Cas10 component is not capable of generating cA₃. In contrast, if cA₃ is detected, the Cas10 component will be suitable for use in any of the methods and/or systems described herein as the generation of cA₃ will activate the NucC nuclease.

In one teaching the Cas10 component may be derived from Vibrio metoecus.

In one teaching, the Cas10 component of any of the systems described herein is not a Thermus thermophilus Cas10.

In view of the above, the disclosure provides a system for the detection of a target nucleic acid, said system comprising
a Vibrio metoecus CRISPR associated protein 10 (Cas10);
a molecule or processed form thereof, capable of binding the target nucleic acid;
a nuclease; and
a reporter system.

Optionally, the system may further comprise ATP.

A type III CRISPR system may comprise a number of additional proteins and a system of this disclosure may further comprise one or more of these additional proteins.

By way of example, a type III CRISPR system may comprise a Csm complex comprising one or more Csm protein(s). Alternatively, a type III CRISPR system may comprise a Cmr complex comprising one or more Cmr proteins. Accordingly, a system of this disclosure may comprise one or more Csm protein(s) and/or one or more Cmr protein(s).

One of skill will appreciate that the number, type and/or stoichiometry of the additional proteins will vary depending on the origin of the Cas10 protein.

The Vibrio metoecus CRISPR system is a type III-B system comprising subunits Cmr1-6 encoded by cmr genes 1-6. In this case, the Cas10 protein is encoded by the cmr2 gene.

Accordingly, the Cmr2 protein may be used as the Cas10 element of a system of this disclosure.

Where the Cas10 element of the system described herein is derived from the type III CRISPR system of Vibrio metoecus, the disclosed system may further comprise one or more additional Cmr proteins.

For example (in addition to Cmr2 (Cas10) a system of this disclosure may further comprise
one or more (for example two, three or more) Cmr1 protein(s); and/or
one or more (for example two, three or more) Cmr3 protein(s); and/or
one or more (for example two, three, four or more) Cmr4 protein(s);
one or more (for example two, three, four or more) Cmr5 protein(s); and/or
one or more (for example two, three or more) Cmr6 protein(s).

The exact protein stoichiometry may vary by, for example ±1 per Cmr protein (and in particular with respect to the Cmr4/5 content).

In one teaching a system may comprise 1×Cmr1 protein; 1×Cmr2 (Cas10) protein; 1×Cmr3 protein; 4×Cmr4 protein; 3×Cmr5 protein and 1×Cmr6 protein.

In view of the above, the disclosed system may comprise:
a Vibrio metoecus CRISPR associated protein 10 (Cas10);
a molecule or processed form thereof, capable of binding the target nucleic acid;
a nuclease;
   a reporter system; and
one or more Cmr1(Cas7) protein(s); and/or
one or more Cmr3 (Cas5) protein(s); and/or
one or more Cmr4 (Cas7) protein(s); and/or
one or more Cmr5 (Cas7) protein(s); and/or
one or more Cmr6 (Cas7) protein(s).

Optionally, the system may further comprise a Cas6 protein and/or ATP.

The reporter system may comprise a nucleic acid (a reporter nucleic acid).

The reporter nucleic acid may comprise a double-stranded DNA (dsDNA).

The reporter system may comprise a DNA molecular beacon.

The reporter nucleic acid may further comprise an optically detectable label, for example a fluorescent molecule (Fluorescein or the like).

The detectable label may be quenched and therefore the reporter nucleic acid may further comprise a quenching molecule. One of skill will appreciate that the quenching molecule will, in use, decrease or inhibit the optically detectable signal (e.g. the fluorescence) from the label. When the reporter nucleic acid is degraded, the label may become de-quenched and optically detectable.

Suitable quenched molecules may include commercially available options such as, for example, Iowa Black® double-stranded DNA substrate. These substrates may be modified to include an optically detectable label such as fluorescein (FAM).

Without wishing to be bound by theory, a binding event between the molecule/processed molecule and any target nucleic acid (for example RNA) is a sample, will activate the Cas10 component of the disclosed system. The generated cA₃ will in turn activate the nuclease component of the system (for example NucC). The activate nuclease then degrades the reporter moiety. For example, an activated nuclease like NucC will degrade a double-stranded DNA based reporter system comprising a quenched fluorescein reporter dye to release a fluorescent signal. Accordingly, the detection of a fluorescent signal from the reporter system will indicate that the system has detected the target nucleic acid and/or that the target nucleic acid is present in the sample.

Use of any of the reporter systems described herein, including, for example DNA based molecular beacons is associated with particular advantages over prior art systems based on RNA molecular beacons. In particular, DNA is more stable and less prone to non-specific degradation. This increases the sensitivity, reliability and accuracy of the system and associated uses/methods described herein.

The reporter system may comprise a capture moiety. The capture moiety may comprise biotin.

The reporter system may be immobilised to a substrate. The immobilisation of the reporter system may occur via the capture moiety. A substrate to which the reporter system may be immobilised may be functionalised so that is captures and immobilised the reporter system. For example, where the reporter system comprises a capture moiety (for example biotin), the substrate may comprise a ligand for that moiety (for example streptavidin). Immobilisation of the reporter to the substrate might occur via binding between the capture moiety and the ligand of the substrate.

The nuclease may be activated by cyclic tri-adenylate (cA₃). Moreover (when activated) the nuclease may degrade the reporter nucleic acid.

The nuclease may not have a CARF domain.

The nuclease may not degrade its cyclic nucleotide activator.

The nuclease may not belong to the Csm6/Csx1 family of ribonucleases.

The nuclease may comprise a NucC nuclease.

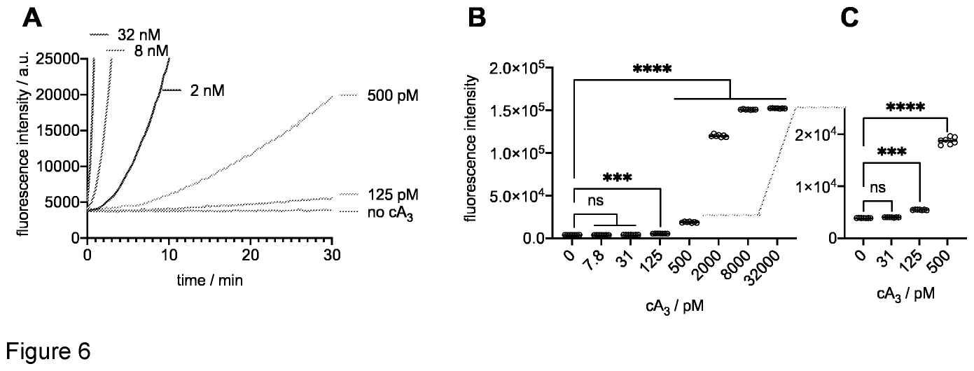

It has been noted that there are a number of advantages associated with the use of the described nucleases, including, for example a NucC nuclease; these include: the fact that NucC is activated by lower concentrations of cyclic nucleotide than prior art nucleases (including, for example, Csm6/Csx1). This enhances sensitivity of the sensitivity of the assay and experimentation has shown that even in the presence of very small amounts of cyclic nucleotide (for example cA₃). In one example, a clear signal was be observed with as little as 10 pM synthetic cAs (see the data presented in FIG. 6).

Moreover, NucC has little or no background activity in the absence of cyclic nucleotide. Again, this enhances sensitivity and reduces the occurrence of false positive results.

Also, unlike prior art technologies (including Csm6/Csx1), NucC does not degrade its cyclic nucleotide activator.

The nuclease may comprise the NucC from Vibrio metoecus. For convenience this nuclease will be referred to as ‘VmeNucC’.

An exemplary VmeNucC sequence is provided below as SEQ ID NO: 3.

(a NucC sequence from Vibro metoecus (WP_
000046098.1))
SEQ ID NO: 3
MAQDWQLSELLENLHADVQHKLTTVRKSFKHSVVKGDGAENVWVDLENQ
YLPERYRASRAFVVDSENQFSEQIDVVIYDRQYSPFIFHYAEQLIIPAE
SVYAVFEVKQTLNKQHIDAARKKVASVRALHRTSLPIPHAGGVHSPREL
IGIIGGLLTLENELKIPDTLMGHLDHDKADKGMLNIGCAADDCFFYYDN
DHQRMQVMQHKKATTAFLFELLSQLQKCGTVPMIDIHAYGKWLTPRISE

It should be noted that orthologous/homologous NucC nucleases may be derived from other microbial species. Any of these NucC nucleases may be used in the methods and systems described herein. Examples of other useful NucC nucleases are provided below as SEQ ID NOS: 4-11.

SEQ ID NO: 4: a NucC sequence Gynuella sunshinyii (WP_044616068.1)
MSDWKLSQLLESLHGDIQHRLKTVRQTIEHPTMKGDGSENVWIGLLNNYLPERYRSSRAF
VVDSNGEFSEQMDVVIYDRQYSPLVFHYEEQLIIPAESVYAVFEVKQTEDKGHIDAAHKK
VASVRKLYRTSMDIVHAGGISKSRTPFSIIGGILALECDLKELETTLKGYLMGADRNDES
KWLTSGCAANRCFFYHDKEHHDIKISQHPKATTAFLFQLLSQLQSCGTVPMLDIHAYGKW
LE
SEQ ID NO: 5: a NunC sequence from a Eubacterium sp. (WP_055150526.1
MDIKSLFNTKQTILESKLSVLLEHPVTKGEHCESAWIDEFRSFLPNKYAVDKGFVFDSKG
NVSDQIDIIIYDALYAPLIFGTDAGEKFITAESVYAVFESKPKINKKTLAYANNKIESVN
VLERSARGVINAGKYYPPRNLTKIIGGILSIDSINIDKIEKHLNLFKYIDLGCAIKNHSF
LVNRESKLSLLHASGEETVLAFFYMILDELYKLGTVAGIDIRNYANANLDNEKFDLDKK
SEQ ID NO: 6: a NunC sequence from a Serratia sp. (WP_021014788.1)
MTNQAKKLSRINGREFLKQSFNLQQQLLASQLNLSRTITHDGTMGEVNESYFLSIIRQYL
PERYSVDRGVVVDSEGQTSDQIDAVIFDRHYTPTLLDQQGHRFIPAEAVYAVLEVKPTIN
KTYLEYAADKAASVRKLYRTSTVIKNIYGTAKPVEHFPIVAGIVAIDVEWQDGLGKAFTE
NLQAVSSDENRKLDCGLAVSGACFDSYDEEIKIRSGENALIFFLERLLGKLQSLGTVPAI
DWRVYIDSLE
SEQ ID NO: 7: a NunC sequence from Pectobacterium carotovorum subsp.
Carotovorum (WP_015841417.1)
MTSQAKELSNINGREFLKNSFDLQQKLLASQLNLSRTITHNGTMGDVNESYFISIIRQYL
PERYSVDRGIVVDSKGQTSDQIDVVIFDRHYTPTLLDQQGHRFIPAEAVYAVLEVKPTIN
KTYLDYAADKAASVRKLYRTSTVIKNIYGKAKPVEHFPIVAGIVAIDVEWQDGLGKAFTA
NLQAISSDGNRKLDCGLAVSGACFDSYDEEIKTKSGENALIFFLFRLLGKLQSLGTVPAI
DWRVYIDSLE
SEQ ID NO: 8: a NunC sequence from Dickeya zeae (WP_012885876.1)
MTNQAKELSKKDGKKYLKNAFKLQQEVLKTQLLQSRAAITHEGVKGDVNEGYFLDIIRQY
LPERYSVDRGIVVNSAGRTSDQIDVIIFDRHYTPTLLNQQGHRLIPAEAVYAVMEVKPII
DASNLDYAADKAASVRSLDRTNMTFRHSGGVGRGRLENIITGIIAIDVDWRDGENSDAFK
KKLQSINQSEDNGHKNLDCGLALSGGCFDFFSKEDEKIKKEEERKKKEEEEKRNRDPQSQ
SEINVKEIFKTIENTQMENLTIRNKEEGALAYFLERLLRELQWLGTAPAIDWNEYVNMLD
HDNEPST
SEQ ID NO: 9: a NunC sequence from Marinomonas mediterranea (WP_013659859.1)
MNNAKTKSDTDGEQYLKDSFQREQELLESKLLFANQNITHNGERGEVNEKHFIEYLRSHL
PKRYSVDSAIVVDSNGKTSNQIDIVVYDNQYTPTLFAQQDFCYVPAEAVYAVIEVKPEVN
RDYIEYAQKQAYSVEALERTSIEITHAGGVYKPKPLFKIITGLIALKTGWQEGLASNSEK
DAIQMPYTNQTRLDFVTSLQGGHEDCFEKGNEHYSQQNHLTFFLERLLSQLQSLGTVPAV
DWNKYANVLTEQSKPD
SEQ ID NO: 10: a NunC sequence from Thioflavicoccus mobilis (WP_015279331.1)
MNSLSRLFASLHEDLERQLSITRESLRHPSAKGDESESIWLALLQNYLPTRYRAERAQVV
DSEGRESDQIDVVVFDRQYSPFIFQLGNQRFVPAESVYAVFEAKQSIGPREITYAKKKIC
SVRALFRTSLPIPHAGGEYEPKPLTPIIGGVLSLESNWNPPLGEPLRKALETNDPMSRID
IGCIASRGLFHSRDGAQTFEFHVGCKTAARFLLRLISELQRIGTVQMIDIMAYERWLVDD
R
SEQ ID NO: 11: a NunC sequence from Paenibacillus popilliae (WP_006285306.1)
MSEPILDHKIIQGIASNYRRLEQAIVDQLRMSSHHHVTSGGFREEMWKQLFEQII PKKYS
VARSVFIIDSEGKVSKEVDLAIFDEQYTPYIFRYGQMKYIPIEAVAVAIQCKSSLSDYDG
IKDWTDSIAKLNTSMKSITRIQSGVVCGEFDEMTENGEFILREGQRPTAQTATRPLIILC
HLDEGFTSKNSLVEYFDFIICPTPTGWLRAFVNKEGHTLEAWYEALNHTNEKYKNVRSPF
GKSSAGNCTLKNYKVHAPDEPENDISLLTLTFQLNQLLMLINNPILFPHQAYVEMENRSL
KE

Also disclosed are the nucleic acid sequences of components for use in any of the systems described herein.

These “system encoding” nucleic acid sequences may be comprised within a vector system for expression in a cell. By way of example, one or more of the nucleic acid sequences encoding one or more components of any of the systems described herein, may be comprised within a bacterial expression vector.

The nucleic acid sequences for any of the system components of this disclosure (for example nucleic acid sequences encoding useful Cas10 elements, useful Cas6 elements, useful reporter sequences, useful Cmr1 proteins, useful Cmr2 proteins, useful Cmr3 proteins, useful Cmr4 proteins, useful Cmr5 proteins, useful Cmr6 proteins, useful nuclease components and sequences encoding the molecules capable of binding the target nucleic acid) may be codon optimised for expression in cell systems, including microbial (bacterial) cells.

An exemplary VmeNucC encoding nucleic sequence may comprise the sequence of SEQ ID
NO: 12.
GCGCCCATGGCACATATGGCCCAGGACTGGCAACTTTCTGAGCTTTTGGAAAACTTACACGCCGATGT
GCAACATAAACTGACCACCGTCCGCAAATCTTTTAAGCATTCGGTAGTTAAAGGTGATGGTGCTGAGA
ACGTCTGGGTTGATTTGTTTAACCAATACTTGCCAGAGCGCTACCGCGCCAGCCGCGCCTTCGTGGTT
GACTCAGAAAATCAATTTTCGGAGCAAATTGACGTAGTCATTTACGACCGTCAGTACAGCCCCTTTAT
CTTTCATTACGCGGAACAGTTAATCATTCCGGCGGAGTCCGTATATGCCGTATTTGAGGTCAAACAAA
CGCTGAATAAGCAGCATATTGACGCTGCACGTAAGAAAGTGGCGTCGGTGCGTGCACTTCACCGTACG
AGTTTGCCCATCCCCCATGCTGGCGGAGTCCACTCCCCACGCGAACTTATTGGTATTATTGGAGGTTT
ACTTACCCTGGAAAACGAACTGAAAATTCCAGACACACTGATGGGACATTTGGATCATGACAAAGCCG
ATAAGGGGATGTTGAACATTGGTTGTGCAGCCGATGATTGTTTCTTCTACTACGACAACGACCATCAA
CGTATGCAGGTCATGCAACACAAAAAGGCGACGACAGCATTCCTGTTCGAGCTGTTGAGTCAGTTGCA
AAAATGTGGGACAGTCCCTATGATTGACATCCACGCCTACGGCAAGTGGTTGACCCCTCGTATTTCTG
AGTGACTCGAGGTCGACCGCG
An exemplary Cas6 encoding nucleic acid sequence may comprise SEQ ID NO: 13.
gcgccatATGGACTCATACATCGATATTCATTTGCGCCCTGACGCCGAGATGAATGAAGCTGAACTTG
GGAGCAAAGTATATACTAAGTTTCATAAAGCATTGGTGAAACTGAATACGAACCAAATCGCGATCTCG
TTTCCAGGGGCTAACCTTAAGCTGGGACAATTATTCCGCGTTCATGGCCCCGTTTCGCTTTTGAATGA
CTTGCAAGGTCTGTGCTGGCTGGGTCCATTATCTGGCTATTGTCAAATCTCAGAGGTGCTTTCCGTAC
CTGAGCAGGTACAATATCGCGTAATCTCTGCAAAACGCCGTAATCTTTCGGCGGCAAAATTGCGTCGC
CTGATTGCACGCGGGTCCATTAACAAGGAGGGGGAACAACGTTACAAAAAAAAAATGTTAAACCAATC
GATCAAGGGACCTTATCTGGATTTGCTGTCCTCTAGCACAGGCCAAAAATATCGTAAATTCTTTGAAT
TTGGTGAAATCCAAGATGTGCCCGTTCTGGGAAATTTCGATACCTATGGCTTATCACTGAAGGCTACA
GTCCCCTGGTTTTGActcgagcgcg
An exemplary VmeRepeat encoding nucleic acid sequence may comprise SEQ ID NO: 14.
cgcgccatggtaaaaatacaatttttaccctaactgactgttgtaacttacttttatagatttattct
ataGTTCACTGCCGCACAGGCAGCTTAGAAAgtgtcttcgtaccttgaagaccaGTTCACTGCCGCAC
AGGCAGCTTAGAAAaatctttatatatcttatggttgcagatctaaaaagttgggattatataaatga
cagtcgacgcgc
SEQ ID NO: 15 provides an example CRISPR array which (in this case) comprises a
promoter, two repeats and a single spacer. When transcribed it generates a guide RNA
molecule
An exemplary Cmr1 encoding nucleic acid sequence may comprise SEQ ID NO: 13
ATGCGCCGTCAGAATAACACCATCGATTTACAGGGACTTAAGAAAGAACTTCTTAACAGCAACAAAGT
CAACGATAAGTGGGAGAGTTATTCCTGCACGCTGGTCACGCCAATGTACGGAGGTGGAGTAAAGGCGG
GAGAGGTGGACAAAGATATGCCTATTCGCGCATCCGCAATCCGTGGCCAGTTACGCTTTTGGTGGCGT
ATTGCGTGTGGGTCCAAGGCACCAGAAGTAATGCGTGAAAATGAGGAAGCTATCTGGGGGGGCATTGG
TGACAAAGCTGCAATTGCCTCTCAGGTTCAGATCCGCGTCATCTCGAAAAATGTAATCATGTCCAACT
TAGTCAGCTCGAAAAAATTGGCAGGTTCTGGGGTCAAGTACGCTCTGGGAGCCGCAGACGAGGCGTAT
TGTTTACCTAGTGGATACAACTTTGTGTTGGAAATCCGCTATAAGGACGATATTACATCCGATCAGAT
CAAGCAGGTGAAAGAATCATTGCGCTGGTGGAGCTCATTCGGCGGGGTAGGGGCCAAAACACGCCGTG
GCTTCGGGGCTGTTGTTGTTGATAGCATTAAAACTATCGAAGCAACGGAGGTGGAAACGATCGGTGGT
AAAATCGCTTTAACTGAGCAGTCCTCAGACTCGGCGCAAGATATGTGGAAAAAGGCCACCGAGCTTTT
ATATAAGTTTCGTCAAGGCCGCGAGTTGGGGCGCAATGAAGGTCAAGGGAACCGTCCTGGCCGTTCAC
GCTGGCCCGAGCCGGATCAATTACGCCGTATGTCAAACAAGCATAAAACAAATCACGAGCCAGAACAT
AAGGCGGGAAATGTGTTTCCACGCGCTGCATTTGGTATGCCAATCATTTTTGATTTTAACGACCGTAG
TCGTACAGAACCATCTACTATGACACTTCTTCCCAAAGATGCCCAACGTATGGCTAGCCCTCTTATTA
TCCGTCCCTACAAAAATGGAGATCAATGGCAAGCAGCAGCATTGTTGCTTCCAAATTGGCAGACGGCA
TTAAACGAGCCCTTGGAGTTGAGTCCTACACCAAACAACGGGACACCTAATCATTGGCCCACCTCGGA
GAATGAGCGTACTCGTTTAGCAGAAATTATTCGCCCTATGGTCGGAAAGAATGGCCAATTGCGTGCGA
ACGATCCACTGTCAGCGTTCCTTGACTTTTTTGAGAATGGCCAATAA
An exemplary Cmr2 encoding nucleic acid sequence may comprise SEQ ID NO: 16
ATGACAGACTACCTGGTTGCCATTTCTATCGGTCCAGTACAGTCGCTGATCGAAGCAGGCCGCCGCGC
GCAAGATCTGTGGTGCGGATCATGGTTACTGAGCGAAGTTGCCCGTGCCGTTGCGTTGAAGCTGCACC
AAACTCAGAACTCGTGCCTGATCTTCCCCAACCCAGAACACCCCCAAGCGGAATTACTTCCGCAATCC
AGCTCCGACGAACCGCGCGCGAACATCGCGAATGTTATCCGCGCTGTTATCTCTGCCGATTCGAAAGA
GGAACTTCAAAATAAGTTAGAAAAAGCAAAAGGAGCGGCCTTCGATCGCCTTTTCGGTATTTTTACGG
AAGTGTTATCGCAAAACGAACTTCAGAATCTTGGCATCGACCGCGCCCGCTGGCAGCAGCAGCAAAAT
GATGTCCTTGAGATCTTTAGTGCTTGGGTTTCTTTAGATAACCACGAATACAACGCTGCGAGCATTCG
CCTTGGTAAGTTGTTGCATGCTCGCAAGTTATCGCGCAATTTCGAGCCTATGAAGGACTGCATGGCTG
CGCTGCATAAAAGTACTCTGGACGGGTCCAACAACACCGTTACCAATAAATTAACTGCGCGTGATGCG
GCTAAAAAGCTGCACATTTCAGATCAAGAACTTCAGCAAAAACGCCGTTTTTTAGGTTTGACGAACGA
AGAGGAATTAGACGCATTGGGAGTAGTGAAACGTCGCGCCGGCAATTTGGAACAGTTCACGCCGTTTT
CGCGCATTGTAGCACATGGATGGTTGAATTCCCTGTCTGATGAACAACGTGCAGGCTTGAAGGTGGCA
TATCAACCCTTCCTTGATAGCGGACACGTAACGCAAGTAAAAGGAAACGATGGCATCTACGCTGATTT
TCCTTTTGATGGTGAGTATCTGTTCTTGAGCCGTCTGGCGCAAGCAGACATTGATCATGATCTGAAAG
ATAAATTACAGAAGCAATTAGCAGCAATCAAGTCATCACCTGTTCCCTATGGTGTTCTGCTTAAGGCG
GATGGAGATCGTATGGGGGATTTACTTAGCAAAGCGGAAGGCAAGCAGCAGTCGAAGGCAATCTCGAA
GGCCTTACACGAATTCGCTACATCCGTTCGTAAAACCTTGCAGGATCATGGGGGTCATGCGATTTATG
CGGGCGGGGACGATGTTCTTGCTTTTGTGCCCCTTGCTCAGGCAATGACCTGCGCGAAACAATTGGCG
GACGATTTCAAGGAAAAAATGAAGGTGATTGCGGCCGAATTAAAACTTAGTGAAGCATTATACCCGAC
ATTAAGCGTCGGTCTGGCTATCGGTCATTTTGTGCAACCCATGCGCCAACTTCGTGCACGTGCCATCG
CTGCGGAAAAACACGCCAAAGGCAACAAAGAGCATAAACCACGCAATGCGTTGGCCATTCACCTTGGT
ATTCGCTCTGGCCATGAGATCACCTGGCGCTGTCGCTGGGATGACGATGAAACCTTAAACGCACTGAC
TGACTTCACGCATGCATTTGCCCAGGGCTGGATGCCCACCCGTATTATGCAAGAGGTTCGCGAGATGG
CAGTGCACTTAAAATGGACAACGGGACAAAAAGAGCTGGCAGGAATTCGTGAGAGCGAGTTAGAACGT
ATGCTTGCACGCGCGGAGTTCAACGTGAGTGTAGAACAGTCAGGTAAGACTCCTGAGCAACTGAAGGA
AAGTAAGAAAGCGGCTTTAGATAAGTTAAAATCACAGTTACGTGCACAAGCATCCGGTTCGCTTGATG
AATTAGCGAACCTGTTGATCTTGGCCCGTTGGTTAAGCGCCAAAACTACAGCGGATATCGGTGGAGAG
GAGTAA
An exemplary Cmr3 encoding nucleic acid sequence may comprise SEQ ID NO: 17
ATGTTATATTATCTTATTGAACCTAAAGATCCCTTAATTATCCGCTCCGGCCGTCCATTCGAAGAGAT
CTCAGATGCGCAGGCGGCGCGTTTCCCACCTCCAAGTACTGTAGCGGGAGCGTTACGCAACATCCATG
CTCGTAGTACAGGTAAAACTTTGGATAACAAGCTTTTGAAACTTGACAACGAATTGCTGAAACTTGCC
GTTACTGGCCCGCTGGCCGTTAAGCGTCCCATTAACGGTGACGCCCCCTCGGAAGAACACATTCTTGT
ACCGAAGCCCGCAGATGTGCAGTACTTCTACGATCAGCAGACGCAGTTGACCCATCTTGTGCGTTCGA
AGCCAATGGCGTTCGCAGAGGGTGAAGGTTGCGACCTTCCGAACGGACTGTTACCGTTATTCGCCGAG
AACGCGCCTGATGGGAAGCCCGTTTCTGGGCCCAACTGGTGGTCCTTCAATGACTTAGCTGCGTGGCG
CAAAGGCCAGTCAGTATCATTCGAGCGTATTTGTCAGAACGGCTGGATGCCTGCGGAACCAGATATCC
GCACACACATTGCTATTAACAACCATTCCCGTAATGTAGAGAGTGGTAAGCTTTTCCAGACGACAGGG
TTGTCCATGTGGCAGCAACGTGCGGATCATCAACCGTTCCCAGACGCATGTGTTAGCATCTTGGCTGG
CATTGACGGTGATATTACCCTTCCGTTGATTAATCTGGGTGGCGAGCGCCGTCTTGCTGAAGTCGAAG
CTTGTACTTTATGGCCTTCTCTGCCCAGCGACTTGGCCCAATCCATCACGAAGGCCAAAGGCTTCACT
TTAACGTTTTTGACCCCAGTGCTTTTTAATTCTGGATGGTTACCGAGTTGGTTAAACGATGAGTTAAT
CGGGACACCTCCCTGCTGTCAGAGTCTGACCGTTAAACTTCGCGCAGCCGCACTTGAACGCTGGATCC
CTCAATCAGGTTGGGACTTGGTAAATAATACACCCCGTGCAGCTCAAAAGATGATTCCGGCAGGTGCG
ACCTATTGGTTTGAGATCGAAGGAGAGGCCACCGACGAGGATATTCGCTCACTTTGGTTAGCCCATTT
TTGTGACGACCCCCAGAGCAACCTGAACGGGTTTGGTCTGGCTTTGCCCGTGGCGTATCAGTTTACTC
TGTAA
An exemplary Cmr4 encoding nucleic acid sequence may comprise SEQ ID NO: 18
ATGAGTTTTCATGTTTACCATCTGTTTTCCCAAACGATCTTACACTGCGGATCGGGCCAGAGTGTCGG
TATTGTTGACCAACCCATCGCGCGCGAGCGCGCATCTAATTTGCCTATCGTTCCTGGATCGACCGTCC
GTGGTGTGCTTAAAGCCTTCATCAGTCACACAGAACAGACTGAGGTAAAACCGACCCTTAGTCAGAGC
TTGTTCGGATATGACAAGCAAGACGGTGAACCAAGTTTTGCAGGCGCTTTGAGTATTACGGACGCTCA
TTTGCTTCTTTTACCCGTCCGCACGGTATACGGTATCCTTGCCTATGCTACATGTCCGTTTATCTTGC
AACGCTATAAAAAAGACCGCAAACTTGATCTTATGGTGCCAGCTCCTGCTGATGAACAAGCACTTCAC
CCGAAAGGAAACCCAAATCACCAAGATAATCTTATGGTCTTAGAAGATTTAGATTTGAAGGTGCAAGA
ATGCGACCATACTCAACAATGGGCTGAGTACATTGCTCAAACACTGTACGCAAAAGACTCAGCTTATT
TCAGCGACATGAGCAAGCGTATCATTGTCTTACCCGATACAGTCTTTTCCTTCCTGGCTGAAACAGCC
ACGGAGATTCGCACGCGCATCCGTATTAACCAGGAGACAGGAGTTGTAGATAATGGCGCACTTTGGAC
AGAAGAAAGCTTGCCGGCTGAATCCGTGTTGTGGGGAGTATACAGTGTCGACGCATCCCGCTTAAACG
ACAAGAACGGAGATAAGCAACTTATGTTTAATGACATTATCAACAATAAGCCCCTGTTGCAAATTGGC
GGCAACATGGGGACGGGTAACGGGTTAGTTCAGTTCATTGCGCAGTCTGCCCGTGGCGAGTAA
An exemplary Cmr5 encoding nucleic acid sequence may comprise SEQ ID NO: 19
ATGCAACCACGCTCGCAGATTGTTGCGACGGCAGCATTCAAGCAAATCAATTCACGTAAGAACAAGTC
CATTGAGGAGAACAAGAAATATGCTACCTTGGCGCATAAATTGCCTACCATGATCTTACAAAACGGGC
TTGCTCAGGCCACCGGGTTCTTGTTAAGTAAAAGCGAAGAACATCATAAAGCTCTGTTAGAAGACTTG
GTGTTGGTGTTTAAACAAGTGGACGCGAAGCTGGCTAACATTGCAAACGCCGAAGCCTTACACGATGC
GATTATCCAAAGTGATTTGCCACAAATTATGCGCATGACTCGTGAGGCCCTTGAAATCGCGGGTTGGT
TACGCCGTTACGTTCAAGGTGTGTTGAAAATTGACGCGACGGGGGAACCGATGGATAAAAAGCCCAAT
GAGAAAGGAGCATGA
An exemplary Cmr6 encoding nucleic acid sequence may comprise SEQ ID NO: 20
ATGGTTCAATTAGTTCGTGACAAATTAAAAGAAGCGTTCGATCATTCCGACAGTATCAACCCCTCGCT
GTTGCTGCAAAAAGGATTGTTAGAGAAGAATAGTGATGCCAAGTCAGACAACAATAAGACGGGGCACT
TGAACAAGATTGTAAAACTGCCTGCCTCGCCTGAATATAAAAATGGCTTTAATCGCTGGTTCGACTTA
ACGCTTGATGAAAATCGCTTTTCCCAGACCGCTATGACGTTGGAAAATCGTCTTTTAATTGGCTTGAC
CGGTCAGGGGGCCTTGGAAACCGGTTGCTCGCTTTCACGTAATTATGGTATGCCGTATATTCCGGGTT
CCTCAGTTAAGGGGGCGGTCCGCGCGTGGGCTAATCAGCATCTTGCTGGACACTCCGACGAGCTGGAG
CAGTTGTTTGGGACGGCCGATTCCGAACAACCCTATCGCGTGTCTGGACTTGTCACATTTCACGACGC
TTGGTGGATTCCTGATCCCGCAAAAAAGGAGCACAAGCCTTTCGTTCTGGATGTAGTGACAACACATC
ACCAGGCATACTACAATGGGACCCAGGCGGAGCCCAGTGATAAAGATAGCCCCATTCCAAACCACCTT
CTGGCCGTACAAGGATCTTTTTTATTCGTATTAGAAGGGGAGTCAGCTGCTATTGAGTTGTGTCAGAC
TCTGTTAGAAAAAGCTCTGGCCAACAACGGCATCGGTGCAAAAACCGCCGCCGGGTACGGCTACATGA
AGGTTGATCCAGCGTTGATGCAACGCCTGCTGGACGAGTATGAAAAGCGTCTGTCACCAGAAGAACGC
GAACACCGTGAGGCTGAAGCTCAACGCCGTAATGAACAACAACTGGAAGCAGCCGCAAAAGCAGAACA
AGCGAAGCCCCCAGTTCAGATCATTGCGGAGTTACGCGCTGGCTATCTGAAACATCGCGATAATGCGG
CTTACCAGTTAAAGGTAGATAATTGGGTCGACACAGCAATCAAAACTTGGACGACGCAGGATCGTACG
TTATTAGCGGAGTGCTTAAAGCAGGTGGGCTACGAGCCGAGTAATAAGAAGAACCCAAATCACTTAAT
TCGCAAAACCCGTTTGCAACAATTGAAGGGCAAATGA

The disclosure provides an expression vector comprising one or more nucleic acid sequences encoding one or more components of any of the systems described herein.

Disclosed is an expression vector comprising one or more nucleic acid sequences including, for example:
a nucleic acid encoding a Cas10 protein; and/or
a nucleic acid encoding a Vme Cas10; and/or
a nucleic acid encoding a molecule which is capable of binding the target nucleic acid; or a molecule which can be processed (by, for example Cas6) into a molecule which is capable of binding the target nucleic acid
a nucleic acid encoding a nuclease; and/or
a nucleic acid encoding a NucC nuclease, for example a VmeNucC; and/or
a nucleic acid encoding a reporter system; and/or
a nucleic acid encoding a Cmr1 protein; and/or
a nucleic acid encoding a Cmr2 protein; and/or
a nucleic acid encoding a Cmr3 protein; and/or
a nucleic acid encoding a Cmr4 protein; and/or
a nucleic acid encoding a Cmr5 protein; and/or
a nucleic acid encoding a Cmr6 protein and/or
a nucleic acid encoding Cas6; and/or
a nucleic acid encoding a CRISPR locus.

A vector encoding a system of this invention may comprise:
a nucleic acid encoding
a nucleic acid encoding a Cas10 protein, for example a Vme Cas10; and/or
a nucleic acid encoding a molecule which is capable of binding the target nucleic acid; or a molecule which can be processed (by for example Cas6) into a molecule which binds the target nucleic acid;
a nucleic acid encoding a nuclease or a NucC nuclease, for example a VmeNucC; and/or
a nucleic acid encoding a reporter system.

The vector may further comprise nucleic acids encoding one or more of:
a Cmr1 protein; and/or
a Cmr2 protein; and/or
a Cmr3 protein; and/or
a Cmr4 protein; and/or
a Cmr5 protein; and/or
a Cmr6 protein.

A vector may also comprise a nucleic acid encoding:
Cas6; and
a CRISPR locus.

In this case, a nucleic acid encoding a CRISPR locus may encode one or more guide RNA(s). an expressed Cas6 element would process the CRISPR locus to yield the one or more guide RNA's which are subsequently assembled into the Cas10 element of the system.

Disclosed is a nucleic acid encoding a Cmr1 protein, a Cmr2 protein, a Cmr3 protein, a Cmr4 protein, a Cmr5 protein, a Cmr6 protein. The nucleic acid may take the form of an expression vector.

The disclosure may also provide a host cell, for example a microbial host cell (like. E. coli or the like) transformed with or comprising any of the nucleic acids and/or (expression) vectors, described herein.

Also disclosed is a diagnostic test or kit or a point of care diagnostic device, comprising a system of this disclosure. For example, the disclosure may provide a lateral flow assay comprising a system of this disclosure. A device of this type may be formed and adapted to receive a sample and the device may be used to detect the presence or absence of a target nucleic acid (for example a RNA) within that sample. The term sample is defined as above.

In one teaching, the device may comprise a substrate (for example a substrate comprising nitrocellulose or a plastic) and the reporter element of the disclosed system may be bound or immobilised to that substrate. As described above, the reporter element may comprise a capture moiety, for example, a biotin moiety, which is used as a means to immobilise it to the substrate.

A diagnostic test or kit or a point of care diagnostic device may comprise or further comprise one or more components of the system described herein. For example, the device may comprise (in addition to any reporter system according to this disclosure—optionally immobilised to a substrate of the device) a:
a CRISPR associated protein 10 (Cas10);
a molecule, capable of binding the target nucleic acid or a molecule which can be processed into a molecule which binds the target nucleic acid (for example one or more guide RNAs which bind to the target nucleic acid);
a nuclease; and
a reporter system.

The molecule/processed molecule (capable of binding the target nucleic acid or a molecule which can be processed into a molecule which binds the target nucleic acid), may comprise a guide RNA. Useful guide RNA(s) will have specificity for a target site within the target nucleic acid.

A sample to be tested for the presence of target nucleic acid may be added to the device and contacted with at least the Cas10 and molecule/processed molecule. When a sample comprising the target nucleic acid is added to a device of this type, binding between the molecule/processed molecule and any target nucleic acid in the sample, may activate the Cas10 component. The device may further comprise any of the nuclease molecules described herein, for example NucC. Any activated Cas10 may in turn activate the nuclease contained within the device. Any activated nuclease may then be brought into contact with the reporter system of the device. As stated, the reporter system may be immobilised to a substrate of the device. The device may comprise a plurality of reporter molecules, all optionally immobilised to a substrate of the device. The nuclease may release at least a part of the reporter molecule so that it can mobilise through the substrate of the device towards a test line. The reporter molecule may comprise a nucleic acid and an activated nuclease may cleave (and therefore release) a reporter moiety from the reporter system and it is this reporter moiety which may then become mobilised through the device towards the test line. A test line of the device may comprise a reporter moiety capture molecule—in other words something, immobilised at the test line, which binds to the reporter moiety to immobilise it at the test line as it moves through the device. The reporter moiety capture molecule may comprise an antibody or some other molecule with affinity or specificity for some part of the reporter moiety.

The reporter moiety may comprise a detectable, for example optically detectable, element. The reporter moiety may further comprise a test line capture element.

Any mobilised reporter moiety may be captured at a test line (via binding between the test line capture element of the reporter moiety and the reporter moiety capture molecule bound or immobilised to or at the test line). As the density or concentration of reporter moiety increases at the test line, the detectable signal increase and the result will be visible—a detectable line indicating that the sample comprises the target nucleic acid.

Of course, if the sample does not contain the target nucleic acid, Cas10 does not become activated, neither does the nuclease and the reporter moiety is not cleaved from the reporter system. As such, there is no accumulation of the reporter moiety at the test line and therefore no detectable signal.

Prior to adding a sample to a device of this disclosure, the sample may be prepared by the addition of buffers. The buffers may comprise agents to facilitate the mobilisation of elements of the sample (for example any target nucleic acid) and/or one or more components of the system described herein, through a device of this disclosure.

A diagnostic test or kit or a point of care diagnostic device may further comprise ATP. The ATP may be contained within a buffer added to the sample and/or the device before, during or after sample addition. As stated elsewhere in this disclosure, the presence of ATP provides a substrate for activated Cas10 to generate cA₃.

The disclosure further provides a diagnostic test or kit or a point of care diagnostic device for use in detecting a target nucleic acid in a sample.

The disclosure further provides a method of detecting a target nucleic acid in a sample, said method comprising contacting a sample with a device described herein and wherein the detection of a signal, for example an optically detectable signal, at the test line of the device, indicates that the sample contains the target nucleic acid.

A sample may be any sample which potentially contains the target nucleic acid, for example a sample of blood (or a fraction thereof, e.g. plasma or serum), urine, saliva or the like. Indeed any of the sample types described earlier can be added to a device of this disclosure.

### DETAILED DESCRIPTION

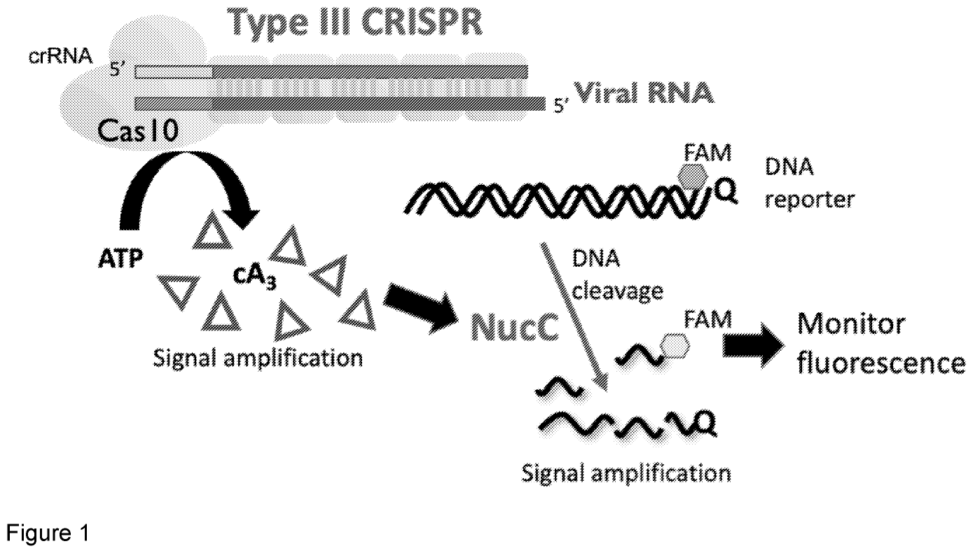

FIG. 1: Schematic representation of the basis for the assay.

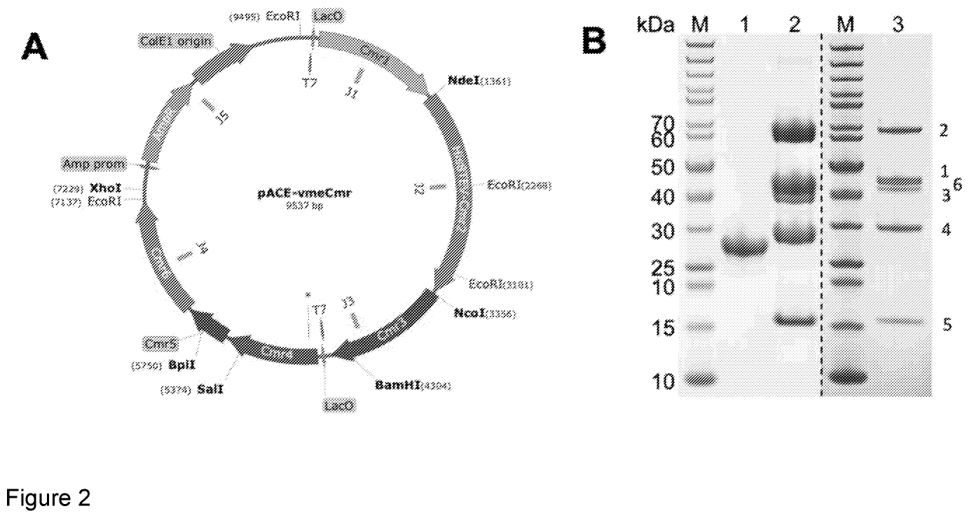

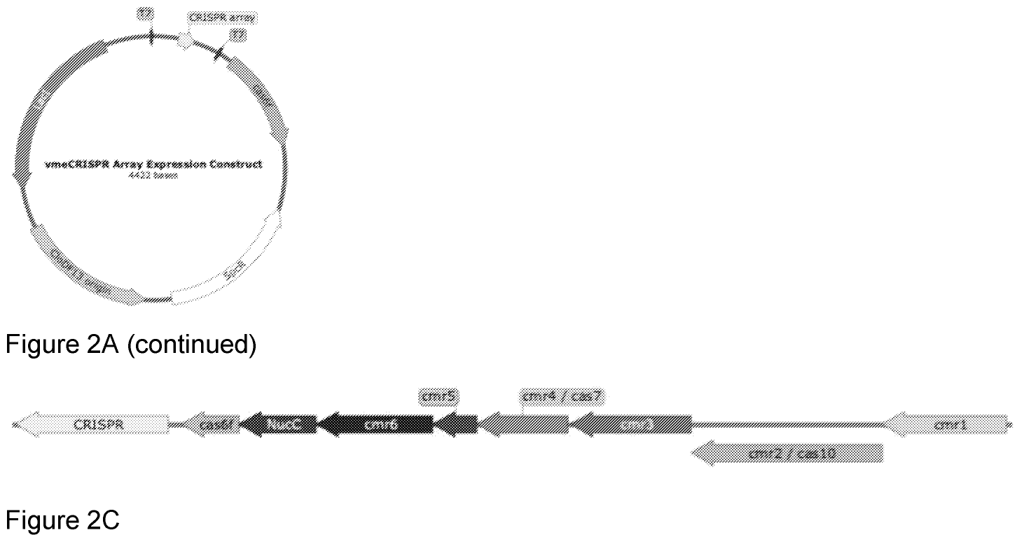

FIG. 2: Plasmid expression construct and SDS-PAGE of purified VmeCmr complexes and vmeNucC used in assays. A. Expression construct for the VmeCmr complex, with the junctions (J1-J5) for Gibson assembly indicated and the expression construct for the CRISPR array and Cas6 protein. B. SDS-PAGE analysis of the purified recombinant proteins. M: PageRuler Unstained (Thermo Scientific); lane 1: VmeNucC; lane 2: VmeCmr wild type; lane 3: VmeCmr with SARS-COV-2 targeting crRNA. The dashed line separates two different gels. The Cmr subunit number is shown on the right of the gel. C. Genomic locus for the type III-B CRISPR locus of Vibrio metoecus.

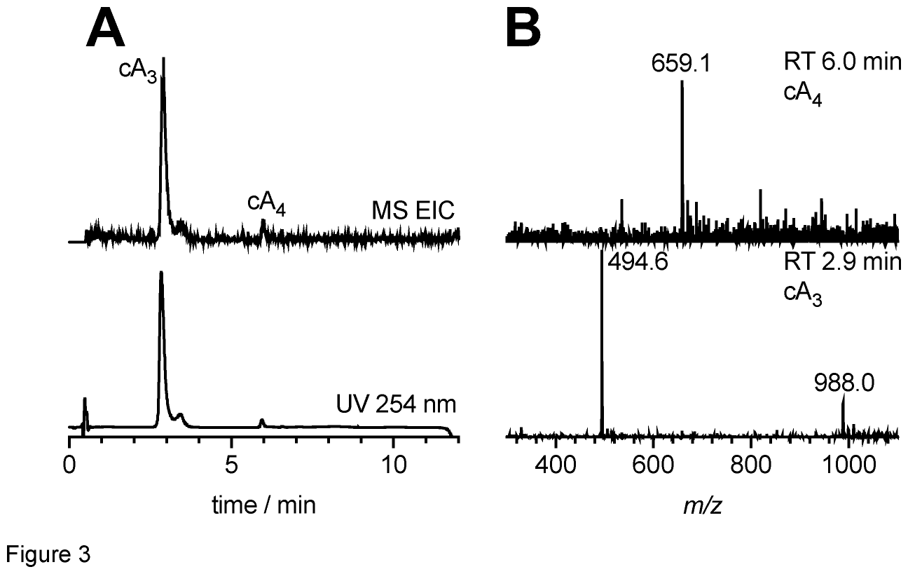

FIG. 3: VmeCmr generates predominantly cA3 when activated by target RNA. A: LC-MS chromatograms with mass and UV monitoring, respectively. The extracted ion chromatogram (EIC) shows all ion species corresponding to cyclic oligoadenylates. B: Mass spectra for products with retention time (RT) 2.9 min and 6.0 min corresponding to cA₃ and cA₄. Expected for cA₃ C₃₀H₃₈N₁₅O₁₈P₃²⁺ m/z 494.7, found 494.6; expected for cA₄ C₄₀H₄₈N₂₀O₂₄P₄²⁺ m/z 659.1, found 659.1;

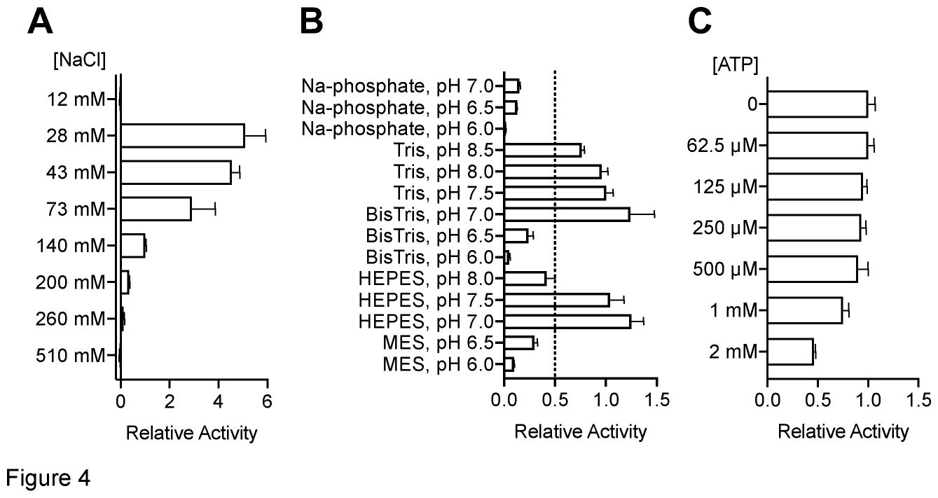

FIG. 4: Factors influencing NucC activity. Relative activities were determined by comparing the relative increase in fluorescence signal over time. A: Effect of NaCl concentration. B: NucC activity in different buffers. C: Effect of ATP on NucC nuclease activity.

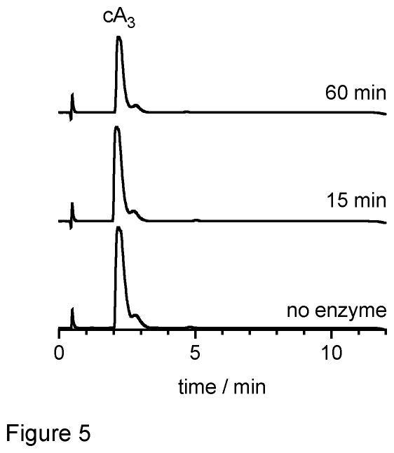

FIG. 5: NucC does not degrade cA₃. LC chromatograms with UV monitoring at 254 nm showing the reaction of 100 μM cA₃ with 0.5 μM NucC under assay conditions after 15 and 60 min. A reaction in which NucC had been omitted was allowed to proceed for 60 min.

FIG. 6: NucC assay using synthetic cA₃ as activator. A: Selected fluorescence signal curves. B: The fluorescence intensities of measurements from 28-30 min reaction time were used for one-way ANOVA analysis. Multiplicity adjusted P values were calculated by Dunnett's multiple comparisons test (N=7). The assay was performed in duplicate. C: selected data from B with expanded y scale. ns: not significant, P value>0.05; ***: P value<0.001; ****: P value<0.0001.

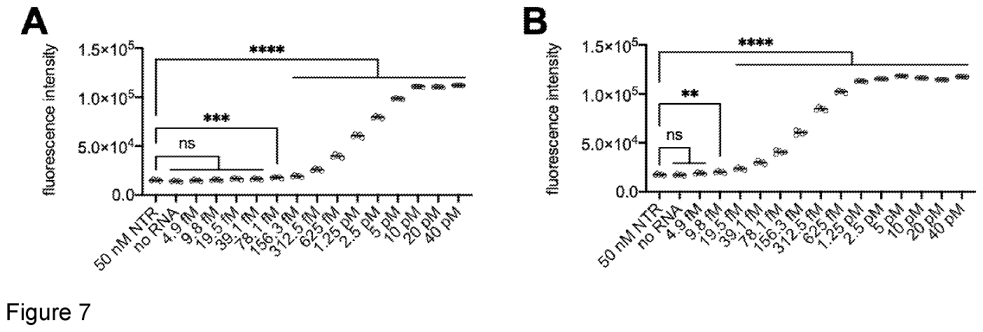

FIG. 7: NucC Limit of Detect (LoD) assay using cA₃ activator generated by wild type Cmr in response to target RNA. The fluorescence intensities of measurements from 28-32 min reaction time were used for one-way ANOVA analysis. Multiplicity adjusted P values were calculated by Dunnett's multiple comparisons test (N=5). At least two independent experiments were performed. A: reference target RNA. B: target RNA with guanosine in the PFS-1 position. NTR: non-target RNA; ns: not significant, P value>0.05; **: P value<0.01; ***:P value<0.001; ****: P value<0.0001.

FIG. 8: Target specificity of vmeCmr. A: Sequences of the crRNA (top) and synthetic target RNA (bottom). B: Topology of the two RNA sequences. C: Effect of the protospacer adjacent site (PFS) in the target RNA on activity. D: Effect of mismatches in the spacer region on activity. Relative activity was determined relative to the reference target RNA sequence shown in A. Nucleotides that differ from the reference sequence are shown in blue.

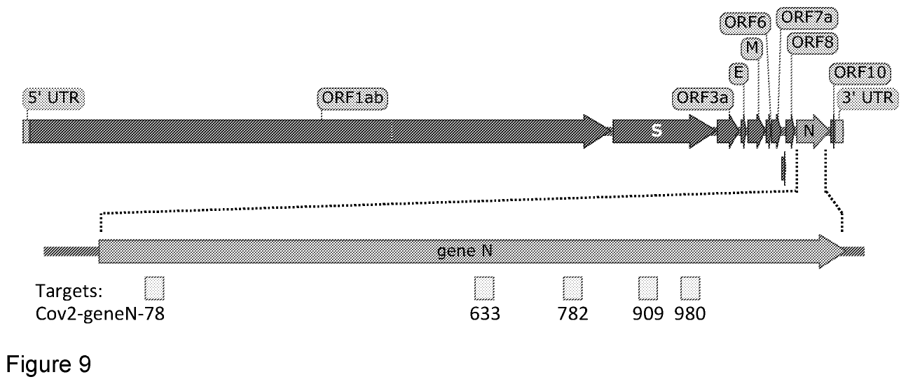

FIG. 9: SARS-Cov-2 genome map and spacer selection. Topology of the SARS-Cov-2 genome (top) and locations of the targets on gene N. Maps were created with SnapGene Viewer.

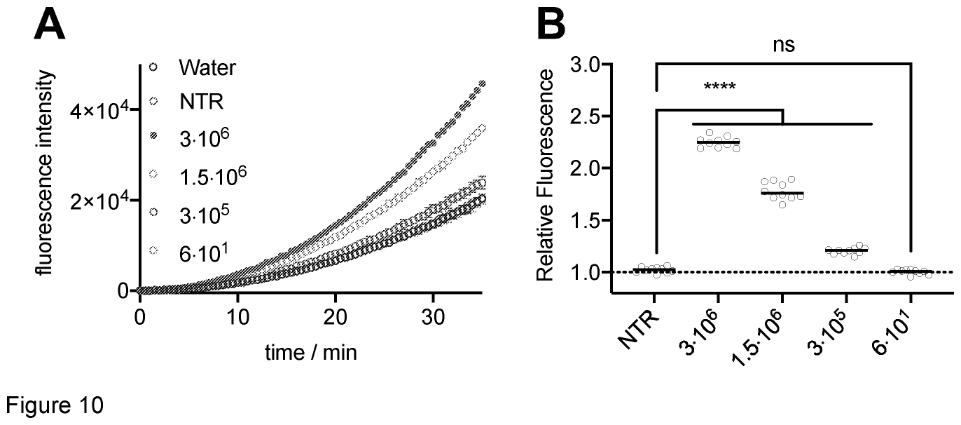

FIG. 10: Detection of SARS-Cov-2 RNA by the coupled Cmr/NucC assay. Nucleic acid extracts from SARS-Cov-2 samples. The extracts are labelled as RNA copies per reaction. A: Fluorescence signal curves. The water, NTR and 6×101 curves overlap. B: Fluorescence intensities relative to the fluorescence signal of the water control. Measurements from 29-31 min reaction time were used for one-way ANOVA analysis. Multiple comparisons against 50 nM NTR (non-target RNA) were performed using the Dunnett's T3 test (N=18). The assay was performed in duplicate. The number of RNA molecules (copies) present in each reaction was calculated using quantitative RT-PCR (39).

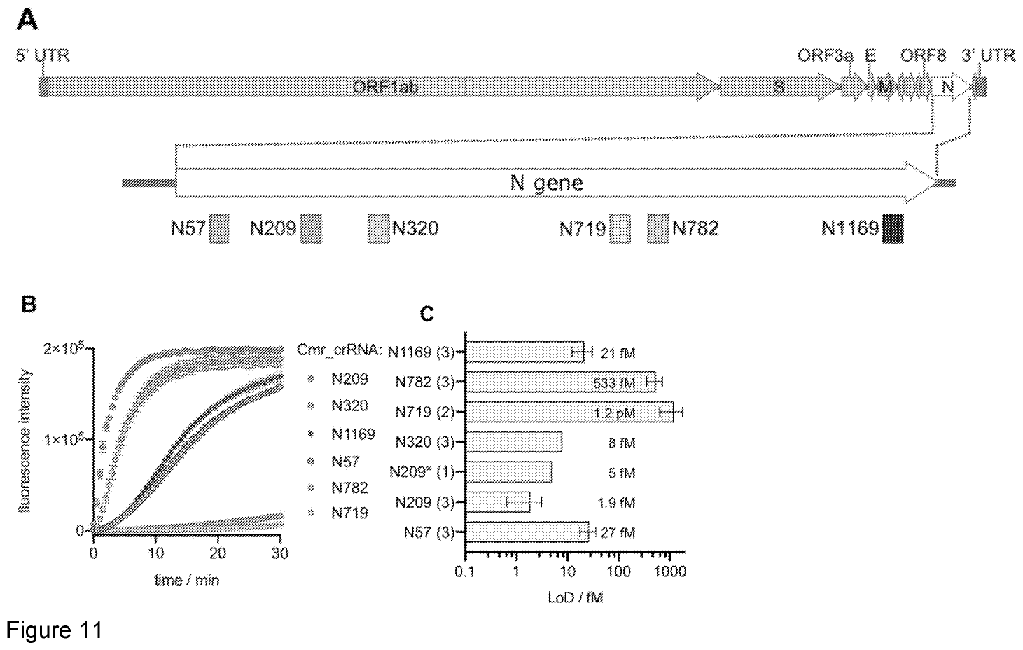

FIG. 11: SARS-COV-2 N gene targeting VmeCmr complexes. A: Further representation of the SARS-COV-2 genome and location of the VmeCmrcrRNA target sites on the N gene. DNA maps were created with SnapGene Viewer. B: VmeCmr complexes charged with crRNAs targeting different positions in the N gene responded in varying degrees to the presence of target RNA. The curves shown were obtained with 2.6 pM N gene transcript and 100 nM VmeCmr complex in the coupled NucC assay. C: LoDs for SARS-COV-2 N gene-targeting VmeCmr complexes. The number of independent experiments is given in brackets next to the crRNA designation. Wild type VmeCmr complexes were used except for N209 (N209*: VmeCmr^{N209}Cmr4 D26A). LoDs were determined from the fluorescence intensities relative to a reference (no RNA) as described in Materials and Methods. Target concentrations with mean fluorescence intensities higher than the mean intensity of the reference plus 10 SDs were regarded as detected. The quoted concentrations correspond to the average LoD value.

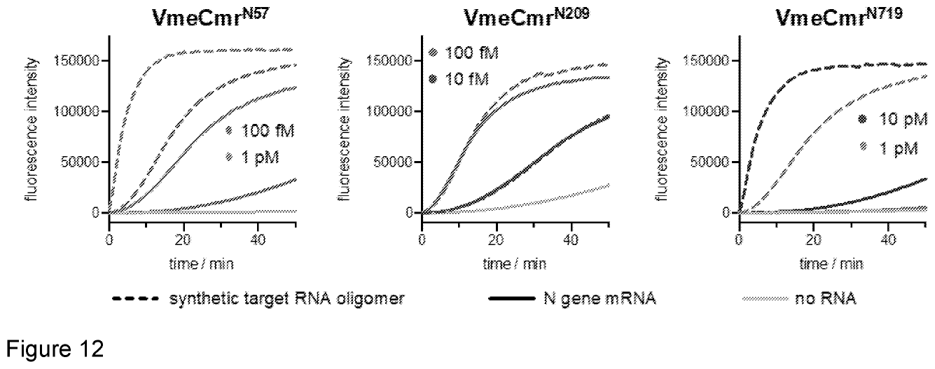

FIG. 12: Comparison of N gene transcript and RNA oligomers as target RNA. The VmeCmr/NucC coupled assay was performed under standard conditions with 50 nM N209, 100 nM N57 or 100 nM N719 and either N gene transcript (solid line) or the 44 nt synthetic RNA (dashed line) containing the cognate target at the indicated concentrations. Identical target concentrations have the same colour.

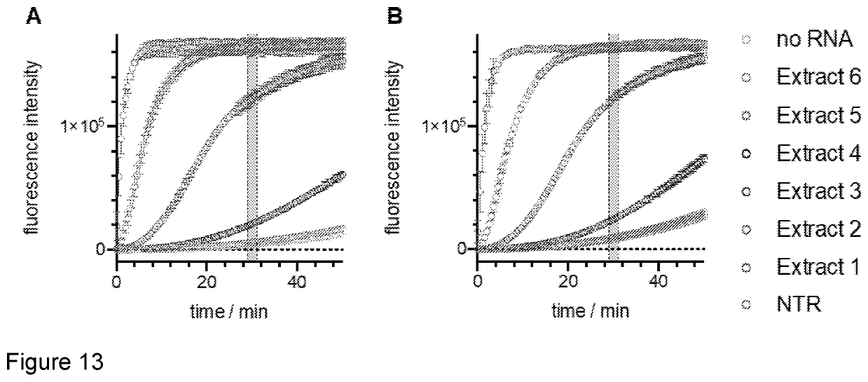

FIG. 13: Detection of SARS-COV-2 N gene in viral extracts. Extracts 1-6 were obtained by extracting a 10-fold serial dilution of viral stocks ranging from 6.106 to 6.101 PFU ml-1, respectively. The assay was performed under standard conditions in triplicate. A: 50 nM wild type VmeCmrN209; B: 25 nM VmeCmrN209 Cmr4 D26A. NTR: non-target RNA.

### MATERIALS AND METHODS

### Cloning

Enzymes were purchased from Thermo Scientific or New England Biolabs and used according to manufacturer's instructions. Oligonucleotides and synthetic genes were obtained from Integrated DNA Technologies (Coralville, Iowa, USA). Synthetic genes were codon-optimized for E. coli and restriction sites for cloning incorporated where necessary. All final constructs were verified by sequencing (GATC Biotech, Eurofins Genomics, DE). Vibrio metoecus nucC was obtained as a G-Block with flanking restriction sites for cloning. After digestion with Ncol and Sall, nucC was ligated into Ncol and Xhol-digested pEV5HisTEV (20) to allow expression as N-terminal His₈-fusion protein.

The expression construct for the Vibrio metoecus Cmr interference complex was assembled using the NEBuilder® HiFi DNA Assembly kit (New England Biolabs) following the Gibson assembly strategy (21). All fragments were PCR-amplified from synthetic genes prior to assembly. The final construct (FIG. 2A) contained a ColE1 origin of replication and ampicillin resistance gene, copied from pACE (MultiColi™, Geneva Biotech, Genève, CH); the expression of cmr1-3 and cmr4-6 was driven by their own T7 promoters. The cmr2 (cas10) gene included a sequence encoding a TEV-cleavable, N-terminal His₈-tag and cmr4 (cas7) had been designed to express as the RNase-dead variant D26A. To obtain the RNase-active (wild type) version, the corresponding codon in the synthetic gene was mutated using primer-directed mutagenesis, the fragment PCR-amplified and then assembled with the remaining fragments as for the cmr4 D26A variant.

The Vibrio metoecus CRISPR array was designed as two repeat sequences flanking two oppositely directed Bpil recognition sites (5′-gtgtcttcgtaccttgaagacca) to allow later insertion of the target/spacer sequence of choice. The synthetic mini-gene also contained flanking Ncol and Sall sites that were used to clone the pre-array into MCS-1 of pCDFDuet™-1 (Novagen, Merck Millipore). Vibrio metoecus cas6f, obtained as G-Block with flanking Ndel and Xhol sites, was subsequently cloned into MCS-2 of the latter construct to give pCDF-notarget_CRISPR. Target/spacer sequences were obtained as synthetic oligonucleotides with a 5′-overhang sequence of 5′-GAAA for the sense strand and 5′-GAAC for the antisense strand. After the two strands were annealed, they were ligated into Bpil-digested pCDF-notarget_CRISPR to give pCDF-target_CRISPR The vmeRepeat and spacer sequences are listed in Table 1. The CRISPR array targeting 5 different sites within the SARS-Cov-2 geneN was constructed by ligation of synthetic DNA fragments into pCDF-notarget_CRISPR. Precise assembly was achieved by creating double-stranded DNA fragments with unique compatible cohesive ends. The sequence for the CRISPR array targeting SARS-Cov-2 geneN is provided in Table 1 and the location of the targets on the gene are shown in FIG. 9. The array was assembled by ligation of synthetic dsDNA oligonucleotides containing compatible cohesive ends. The array was inserted into pCDF-notarget_CRISPR replacing the existing repeats.

### Protein Production and Purification

VmeCmr complex. E. coli BL21 Star™ (DE3) cells (Invitrogen) were co-transformed with pACE-vmeCmr and pCDF-pUC_CRISPR. Overnight cultures were diluted 100-fold into LB containing 100 μg ml⁻¹ ampicillin and 50 μg ml⁻¹ spectinomycin, incubated at 37° C., 180 rpm until the OD₆₀₀ reached 0.8. After induction with 200 μM IPTG, incubation was continued at 27° C. overnight. Cells were harvested by centrifugation and pellets stored at −20° C. Cells were resuspended in lysis buffer (50 mM Tris-HCl, 500 mM NaCl, 20 mM imidazole, 10% glycerol, pH 7.5) and lysed by sonication. The cleared lysate was loaded onto a pre-equilibrated HisTrap Crude FF (GE Healthcare) column, washed with lysis buffer and eluted in a gradient with increasing imidazole concentration (to 0.5 M). Cmr complex-containing fractions were pooled, dialysed at 4° C. overnight in the presence TEV protease against lysis buffer without imidazole. The protein solution was passed through the HisTrap Crude FF column a second time, the flow-through was concentrated using an Amicon Ultracentrifugal filter (30 kDa MWCO, Merck-Millipore) and further purified by size exclusion chromatography (HiPrep™ 16/60 Sephacryl® pg 300 HR, GE Healthcare) using 20 mM Tris-HCl, 500 mM NaCl, 10% glycerol, pH 7.5 as mobile phase. Cmr-containing fractions were concentrated as before and simultaneously buffer-exchanged against 20 mM Tris-HCl, 250 mM NaCl, 10% glycerol, pH 7.5. Single-use aliquots were flash-frozen and stored at −70° C. Protein concentrations were determined by UV quantitation (NanoDrop 2000, Thermo Scientific) using calculated extinction coefficients (ExPASy, ProtParam software for protein; AAT Bioquest for nucleic acids). The concentration of Cmr complex was estimated using an extinction coefficient of 610,240 M⁻¹ cm⁻¹, which was obtained by adding the values for the protein component in Cmr11 Cmr21 Cmr31 Cmr44 Cmr53 Cmr61 stoichiometry and an estimated value for the crRNA (calculated 396,900 M⁻¹ cm⁻¹ at 260 nm, used 200,000 M⁻¹ cm⁻¹ for 254 nm).

VmeNucC. The production and purification of vmeNucC was the same as for the vmeCmr complex with the following two exceptions: E. coli C43(DE3) was used as the expression host, and size exclusion chromatography was carried out on a HiLoad® 16/60 Superdex® pg 200 column (GE Healthcare) using 20 mM Tris-HCl, 250 mM NaCl, 10% glycerol, pH 7.5 as mobile phase.

TABLE 1
repeat and target/spacer sequences

| | | |
|---|---|---|
| vmeRepeat | 5′-GTTCACTGCCGCACAGGCAGCTTAGAAA | |
| vmepUCspacer- | 5′-GAAAgaattcgagctcggtacccggggatcctctag | pUC target spacer |
| top | | (Vibrio Cmr) |
| vmepUCspacer- | 5′-GAACctagaggatccccgggtaccgagctcgaattc | pUC target spacer |
| btm | | (Vibrio Cmr) |
| Target RNA | 5′- | RNA |
| | GACUcuagaggauccccggguaccgagcucgaauucAAA | |
| | GAUUC | |
| Non-target RNA | 5′- | RNA |
| (NTR) | aggguauuauuuguuuguuucuucuaaacuauaagcuagu | |
| | ucuggaga | |
| NucC substrate | 5′-FAM-agtgttacattatccaccatggcgagcttt | DNA |
| 5′-FAM | | |
| NucC substrate | 5′-aaagctcgccatggtggataatgtaacact-Iowa Black | DNA |
| 3′-lowaBlack | | |
| GeneN- | 5′- | |
| targeting | GTTCACTGCCGCACAGGCAGCTTAGAAAacgct | |
| CRISPR array | gaagcgctgggggcaaattgtgcaattGTTCACTGCCGC | |
| | ACAGGCAGCTTAGAAAatcgcgccccactgcgttctcca | |
| | ttctggttaGTTCACTGCCGCACAGGCAGCTTAGA | |
| | AAgcaaagcaagagcagcatcaccgccattgccaGTTCAC | |
| | TGCCGCACAGGCAGCTTAGAAAgatggcacctgtgt | |
| | aggtcaaccacgttcccgGTTCACTGCCGCACAGGC | |
| | AGCTTAGAAAtgttacattgtatgctttagtggcagtacgttGT | |
| | TCACTGCCGCACAGGCAGCTTAGAAA | |
| Cov2-geneN-78 | ′5- | synthetic RNA |
| | GCAGuaaccagaauggagaacgcaguggggcgcgauCA | target |
| | AAACAA | |
| Cov2-geneN- | 5′- | synthetic RNA |
| 633 | UGGCuggcaauggcggugaugcugcucuugcuuugcUG | target |
| | CUGCUU | |
| Cov2-geneN- | 5′- | synthetic RNA |
| 782 | CAAAaacguacugccacuaaagcauacaauguaacaCAA | target |
| | GCUUU | |
| Cov2-geneN- | 5′- | synthetic RNA |
| 909 | CGCAaauugcacaauuugcccccagcgcuucagcguUC | target |
| | UUCGGA | |
| Cov2-geneN- | 5′- | synthetic RNA |
| 980 | CCUUcgggaacgugguugaccuacacaggugccaucAAA | target |
| | UUGGA | |

### Nuclease assay

All assays were performed on a FluoStar Omega plate reader (BMG Labtech) using fluorescence detection (λₑₓ/ₑₘ 544/590 nm) and Corning black, non-binding half-area 96-well plates. A typical reaction for Vibrio metoecus Cmr contained 250 nM wild type VmeCmr complex in 12.5 mM Tris-HCl, pH 8.0, 10-20 mM NaCl, 10 mM MgCl₂, 10% glycerol, 500 μM ATP, 125 nM FAM : Iowa Black® double-stranded DNA substrate (Table 1) and varying concentrations of target or control RNA. The reaction was incubated at 37° C. for 10 min to allow cyclic oligoadenylate synthesis before addition of 250 nM NucC to start the nuclease reaction. Fluorescence was measured in 30 s intervals at 37° C. for up to 60 min. Statistical analyses were performed with Prism 8 (GraphPad). To determine limit of detection, the means of fluorescence intensities ranging between 28 and 32 min was analysed by one-way ANOVA and for multiple comparisons the Brown-Forsythe and Welch ANOVA test was applied. Multiplicity adjusted p values <0.05 were taken to be statistically significant. Relative activities between reaction conditions was determined by comparing the relative rates of the reactions. Rates were assumed to be proportional to the change in fluorescence signal over time and calculated from the slopes of linear regression analysis. Target specificity was analysed by extracting the time point (Tₜ) when the fluorescence intensity crossed a threshold value. The threshold value was set to ⅛^{th} of the maximal measured fluorescence signal of the reference target. Individual Tₜ values were subtracted from the Tₜ value obtained in the absence of RNA to give DeltaTₜ values. Finally, DeltaTₜ values were normalized to the DeltaTₜ value of the reference target.

### SARS-COV-2 RNA Preparation

SARS-COV-2 virus was purified from infected VeroE6 cells and the viral RNA isolated according to established procedures (38).

### RESULTS

### Cloning, Expression and Purification

To investigate the utility of the VmeCmr/NucC system for specific RNA detection, we built two plasmid constructs, one expressing synthetic versions of the codon optimised cmr1-6 genes (FIG. 2A) and a second encoding Cas6 and a mini-CRISPR array (see materials and methods). This system can be programmed to detect any RNA sequence by changing the spacer sequence in the CRISPR array and can be multiplexed to detect any desired number of different RNA sequences by adding spacer sequences. Initially we programmed the system using a single spacer that matches a synthetic target RNA species used previously (22). The VmeCmr complex expressed at a high level in E. coli and could be purified in mg quantities (FIG. 2B). We also expressed a codon-optimised gene encoding VmeNucC and purified the recombinant protein (FIG. 2B). We confirmed that VmeCmr generates predominantly cA₃ when activated by target RNA, with much lower levels of cA₄ made, as expected for a system coupled to NucC (FIG. 3).

### VmeNucC Functions as a cAs Activated Nuclease That Can Generate a Fluorescent Signal In Vitro

To characterise the nuclease activity of VmeNucC, we used a synthetic DNA duplex of 30 bp with a fluorescein reporter dye quenched by an IOWA Black quencher and monitored the fluorescent signal generated by NucC using a FluoStar Omega plate reader. VmeNucC was most active in 28-70 mM NaCl (FIG. 4A). Similarly, a good range of buffers with pH of 7.0-8.0 supported NucC activity satisfactorily. The best buffers were BisTris, PH 7.0, HEPES, pH 7.0-7.5, and Tris-HCl, pH 7.5-8.5 (FIG. 4B). One major limitation of Csx1/Csm6 family enzymes is their inhibition by ATP (5,6), which is an essential component of detection assays. ATP only had a minor adverse effect on NucC at concentrations above 500 μM, with 50% of NucC activity retained at an ATP concentration of 2 mM (FIG. 4C).

### NucC Does Not Degrade its cA₃ Activator

Some Csx1/Csm6 family proteins have an intrinsic “ring nuclease” activity for the degradation of their cOA activator—an activity that is probably important in vivo for the control of the CRISPR mediated immune response (9). In the context of assay development however, this is an undesirable characteristic. Furthermore, it is difficult to remove this activity from Csx1/Csm6 family proteins as the activity is closely linked with cOA binding and/or with the HEPN ribonuclease activity (18, 19). To determine whether NucC degrades its cA₃ activator, we incubated 100 μM cA₃ with 0.5 M NucC in reaction buffer for 60 min and monitored the products by liquid chromatography and UV detection (FIG. 5) after removal of the enzyme by ultracentrifugation (MWCO 3 kDa). The assay showed no significant depletion of cA₃ and no trace of any degradation products, suggesting that NucC does not degrade its own activator. This is perhaps not surprising given that NucC is a double stranded DNase and the activator is a cyclic ribonucleotide.

### NucC is Activated by Very Low Concentrations of cA₃

A major limitation in any approach utilising a cOA-activated nuclease in sensitive RNA detection assay coupled to Cas10 is the affinity of the nuclease for the activator. The dissociation constant of >=100 nM observed for Csx1/Csm6 family proteins binding to cOA (9, 16, 17) means that this concentration of cOA is required for half-maximal activation of the nuclease. As the cOA level generated by Cas10 is directly proportional to the RNA present in the sample (8,9), this places intrinsic limitations on the sensitivity of the method. As a rule of thumb, 100 nM cOA is generated by Cas10 in response to 0.1-1 nM RNA.

By titrating the concentration of cA₃ in the assay, we investigated the limits of detection of the fluorescent signal generated by 250 nM VmeNucC trimer. Clear signals were observed after 30 min incubation with as little as 10 PM cA₃ activator (FIG. 6).

### Coupling of NucC Activation With Target RNA Detection

Having confirmed that Vme NucC is activated by synthetic 3′,5′-cA₃ and can generate a fluorescent signal, we proceeded to couple the V. meteocus type III system by using Target RNA complementary to the crRNA spacer sequence of the ribonucleoprotein (RNP) complex (see Table 1 for sequences) to activate the cyclase activity of Cas10, coupling cA₃ production to a NucC mediated fluorescent readout. We first titrated target RNA concentrations to establish the limits of RNA detection using the NucC-coupled fluorogenic assay. For wild-type VmeCmr, RNA target concentrations at the femtomolar level produced a fluorescent signal within 30 min of initiating the assay that was statistically significant by comparison with a control reaction using 50 nM non-target RNA. RNA target concentrations in the mid-picomolar range, on the other hand, produced a very fast signal that reached its maximum within 10 min. The limit of detection ranged from 10-80 fM of target RNA depending on the nature of the nucleotide at the −1 position (FIG. 7) as discussed below. By comparison, an assay system developed using Thermus thermophilus Cmr coupled with the Csx1/Csm6 family ribonuclease TTHB144 had a limit of detection of 1 nM RNA (1). Our assay is therefore five orders of magnitude more sensitive.

### Specificity of Target RNA Detection

Type III CRISPR systems must avoid inappropriate activation by RNA targets such as anti-sense RNAs transcribed from the CRISPR locus, which could cause toxicity or even cell death. To achieve this, Type III systems sense mispairing of RNA at the Protospacer flanking site (PFS), which is immediately 3′ of the RNA duplex formed between the target RNA and the crRNA, corresponding to the repeat-derived 5′-handle of the crRNA. When an anti-sense CRISPR RNA binds, it base-pairs along the length of the PFS, preventing activation of the HD nuclease or cyclase activities of Cas10 (6-8,23-27). A detailed investigation of the Type III-B system from Thermatoga maritima (TmaCmr) (28) revealed that the three nucleotides at positions −1 to −3 of the PFS are crucial in regulating Cas10 activity, consistent with observations in type III-A systems (29-31) and furthermore that guanine at position −1 is sensed directly, rather than via base-pairing, to keep the complex in an inactive state (28).

We first tested the importance of the PFS for VmeCmr activity (FIG. 8) by changing one or more nucleotides in the target RNA sequence. At position −1, all four nucleotides were well tolerated but a Guanine at position −1 resulted in a higher level of activation of VmeCmr. This observation suggests that selection of target sequences with a G at position −1 could be advantageous for assay sensitivity. Introduction of a U:A base pair at position −2 reduced activity significantly, but equivalent base pairs introduced singly at positions −3 to −5 had only a modest impact on activity (FIG. 8C). However, introduction of a run of three base-pairs at positions −2 to −4 virtually abolished VmeCmr activity, emphasising the importance of targets which do not base pair with the crRNA in this region, as observed previously (29-31).

Type III CRISPR systems are tolerant of extensive mis-pairing between crRNA and target RNA, a factor which is postulated to limit viral escape by mutation (8,28,32-35). The bound target RNA can be divided into 5 bp segments followed by a sixth nucleotide that is flipped out of the duplex by the Cas7 subunit (30,31,36,37). Segment 1, matching the 5′-end of the spacer sequence, adjacent to the PFS, is particularly important for Cas10 activation (8,28), analogous to the “seed” region next to the protospacer adjacent motif in type I, II and V systems. The tolerance of mismatches in between the target RNA and crRNA may help type III CRISPR systems to target rapidly evolving phage (32), but in the context of assay development it is important to demonstrate specific nucleic acid detection.

To investigate this, we changed single nucleotides in the target RNA at positions 1 to 9, making the nucleotide at each position identical to, rather than complementary to, the crRNA sequence (FIG. 8D). At the lowest concentration of target RNA tested (5 pM), single mutations at positions 1 to 5 all resulted in a significant decrease in cA₃ production and therefore NucC activity, confirming the importance of segment 1 observed for other type III systems (8,23,28). Mutation at position 6, which is not base-paired in the ribonucleoprotein complex, had no effect on the cyclase activity, as expected, and there were minor reductions in cyclase activity for positions 7 and 8 in segment 2. VmeCmr is thus exquisitely sensitive to single nucleotide mismatches in segment 1, which contrasts with the findings for TmaCmr, where significant effects on Cas10 activity were only observed when four or five nucleotides were mutated simultaneously (28). A SNP at position +1 or +3 resulted in almost complete abolition of the fluorescent signal.

To conclude this section, we observe that target RNA is detected specifically by VmeCmr, with the seed region in segment 1 (positions 1-5 in FIG. 8) particularly important and contributions also arising from segment 2. Selection of targets with a G at position −1 may also contribute to enhanced VmeCmr activity. Coupled with previous observations, we conclude that, with careful design of targeting sequences, VmeCmr is suitable for highly specific RNA detection.

### Multiplexed Detection of the SARS-COV-2 N Gene RNA by VmeCmr

To programme VmeCmr to detect the SARS-COV-2 RNA specifically, we designed a mini-CRISPR with 5 spacers matching five different sequences in the N gene of the virus (FIG. 9). This ribonucleoprotein complex was expressed and purified as before (FIG. 1).

This version of the Cas10 assay was tested with RNA purified from SARS-COV-2 with a defined number of plaque forming units (PFU) (38). The RNA present in these samples was quantified by q-RT-PCR using primers specific for SARS-COV-2 (Altona Diagnostics Realstar SARS-COV-2 RT-PCR kit) to determine the number of viral copies present in the samples (39). Our data shows that a reaction with 3×105 copies of the viral target RNA can be detected directly by our assay without any amplification step (FIG. 10). This compares with a LoD using the T. thermophilus type III system of 1×10⁷ (2) or 1.2×10¹⁰ copies detected (1).

### Example 2: Sensitive Detection of the SARS-COV-2 N Gene RNA by VmeCmr/NucC.

Further experiments were conducted to determine the sensitivity of the VmeCmr/NucC system for detection of larger RNA species. The SARS-COV-2 N gene was used as an exemplar. To programme VmeCmr to detect the SARS-COV-2 RNA specifically, we designed, expressed and purified six different VmeCmr complexes carrying guide RNAs designed to match a range of positions in the SARS-COV-2 N gene (FIG. 11A). The VmeCmr constructs were named according to the first nucleotide of the N gene matching the crRNA (FIG. 11A and Table 2 below). Each was designed to have a G at position −1 of the PFS, as that provided the highest activity with the reference target set (FIG. 8C). A ˜1250 nt in vitro transcript of the N gene was generated to serve as target RNA for the assays.

It was noted that the background activity in the absence of any added RNA target became significant for some of the six VmeCmr constructs. Therefore all complexes were subject to a further purification step by heparin chromatography, which significantly reduced the background activity without affecting the signal generated by the activated complex. Accordingly, all further assays were conducted with VmeCmr complexes that had undergone the additional heparin purification step.

As is clear from FIG. 11B, a wide range of activities for the six different complexes was observed. The best ones generated a large fluorescent signal within 1-2 min when activated by 2.6 pM transcript while the least sensitive constructs gave only a marginal signal. The difference in activity was reflected in the LoD obtained for each complex (FIG. 11C). The most sensitive complexes were N209 and N320 with LoDs of 1.9 fM and 8 fM, respectively. The N719 complex was the poorest with an LoD of 1.2 pM; thus, the measured LoDs spanned 3 orders of magnitude for the six investigated VmeCmr complexes. The Cmr4 D26A variant of the N209 targeting VmeCmr complex was also tested. This mutation targets the Cmr4 (Cas7) active site and is known to prevent degradation of the target RNA in all other type III systems studied (40-42. 26, 8, 30, 43, 41). It has previously been shown that preventing target RNA degradation in type III CRISPR-Cas complexes leads to increased cOA production (45,8,43) which in turn would be expected to lower the target RNA concentration required to trigger NucC activity. For VmeCmrN209 Cmr4 D26A, however, no improvement in sensitivity was observed.

TABLE 2

| | SARS-CoV-2 target RNA Sequence |
|---|---|
| Sequence Name | (5′ to 3′) |
| N209 target | AAGGCGUUCCAAUUAACACCAAUAGCAGUCCA |
| N320 target | GAUGGUAUUUCUACUACCUAGGAACUGGGCCA |

### REFERENCES

1. Steens, J. A., Zhu, Y., Taylor, D. W., Bravo, J. P. K., Prinsen, S. H. P., Schoen, C. D., Keijser, B. J. F., Ossendrijver, M., Hofstra, L. M., Brouns, S. J. et al. (2021) SCOPE: flexible targeting and stringent CARF activationenables type III CRISPR-Cas diagnostics. BioRxiv.
2. Santiago-Frangos, A., Hall, L. N., Nemudraia, A., Nemudryi, A. A., Krishna, P., Wiegand, T., Wilkinson, R. A., Snyder, D. T., Hedges, J. F., Jutila, M. A. et al. (2020) Intrinsic Signal Amplification by Type-III CRISPR-Cas Systems Provides a Sequence-Specific Viral Diagnostic. MedRxiv, https://doi.org/10.1101/2020.1110.1114.20212670.
3. Lau, R. K., Ye, Q., Birkholz, E. A., Berg, K. R., Patel, L., Mathews, I. T., Watrous, J. D., Ego, K., Whiteley, A. T., Lowey, B. et al. (2020) Structure and Mechanism of a Cyclic Trinucleotide-Activated Bacterial Endonuclease Mediating Bacteriophage Immunity. Mol. Cell, 77, 723-733.
4. Chen, J. S., Ma, E., Harrington, L. B., Da Costa, M., Tian, X., Palefsky, J. M. and Doudna, J. A. (2018) CRISPR-Cas12a target binding unleashes indiscriminate single-stranded DNase activity. Science, 360, 436-439.
5. Gootenberg, J. S., Abudayyeh, O. O., Kellner, M. J., Joung, J., Collins, J. J. and Zhang, F. (2018) Multiplexed and portable nucleic acid detection platform with Cas13, Cas 12a, and Csm6. Science, 360, 439-444.
6. Kazlauskiene, M., Kostiuk, G., Venclovas, C., Tamulaitis, G. and Siksnys, V. (2017) A cyclic oligonucleotide signaling pathway in type III CRISPR-Cas systems. Science, 357, 605-609.
7. Niewoehner, O., Garcia-Doval, C., Rostol, J. T., Berk, C., Schwede, F., Bigler, L., Hall, J., Marraffini, L. A. and Jinek, M. (2017) Type III CRISPR-Cas systems produce cyclic oligoadenylate second messengers. Nature, 548, 543-548.
8. Rouillon, C., Athukoralage, J. S., Graham, S., Grüschow, S. and White, M. F. (2018) Control of cyclic oligoadenylate synthesis in a type III CRISPR system. eLife, 7, e36734.
9. Athukoralage, J. S., Graham, S., Rouillon, C., Grüschow, S., Czekster, C. M. and White, M. F. (2020) The dynamic interplay of host and viral enzymes in type III CRISPR-mediated cyclic nucleotide signalling. eLife, 2020;9:e55852.
10. Makarova, K. S., Timinskas, A., Wolf, Y. I., Gussow, A. B., Siksnys, V., Venclovas, C. and Koonin, E. V. (2020) Evolutionary and functional classification of the CARF domain superfamily, key sensors in prokaryotic antivirus defense. Nucl. Acids Res., 48, 8828-8847.
11. Foster, K., Kalter, J., Woodside, W., Terns, R. M. and Terns, M. P. (2019) The ribonuclease activity of Csm6 is required for anti-plasmid immunity by Type III-A CRISPR-Cas systems. RNA Biol, 16, 449-460.
12. Jia, N., Jones, R., Yang, G., Ouerfelli, O. and Patel, D. J. (2019) CRISPR-Cas III-A Csm6 CARF Domain Is a Ring Nuclease Triggering Stepwise cA4 Cleavage with ApA>p Formation Terminating RNase Activity. Mol. Cell, 75, 944-956 e946.
13. Niewoehner, O. and Jinek, M. (2016) Structural basis for the endoribonuclease activity of the type III-A CRISPR-associated protein Csm6. RNA, 22, 318-329.
14. Joung, J., Ladha, A., Saito, M., Kim, N. G., Woolley, A. E., Segel, M., Barretto, R. P. J., Ranu, A., Macrae, R. K., Faure, G. et al. (2020) Detection of SARS-COV-2 with SHERLOCK One-Pot Testing. N Engl J Med, 383, 1492-1494.
15. Kellner, M. J., Koob, J. G., Gootenberg, J. S., Abudayyeh, O. O. and Zhang, F. (2019) SHERLOCK: nucleic acid detection with CRISPR nucleases. Nature protocols, 14, 2986-3012.
16. Samolygo, A., Athukoralage, J. S., Graham, S. and White, M. F. (2020) Fuse to defuse: a self-limiting ribonuclease-ring nuclease fusion for type III CRISPR defence. Nucl. Acids Res., 48, 6149-6156.
17. Athukoralage, J. S., Graham, S., Grüschow, S., Rouillon, C. and White, M. F. (2019) A type III CRISPR ancillary ribonuclease degrades its cyclic oligoadenylate activator. J. Mol. Biol., 431, 2894-2899.
18. Molina, R., Stella, S., Feng, M., Sofos, N., Jauniskis, V., Pozdnyakova, I., Lopez-Mendez, B., She, Q. and Montoya, G. (2019) Structure of Csx1-cOA4 complex reveals the basis of RNA decay in Type III-B CRISPR-Cas. Nature communications, 10, 4302.
19. Garcia-Doval, C., Schwede, F., Berk, C., Rostol, J. T., Niewoehner, O., Tejero, O., Hall, J., Marraffini, L. A. and Jinek, M. (2020) Activation and self-inactivation mechanisms of the cyclic oligoadenylate-dependent CRISPR ribonuclease Csm6. Nature communications, 11, 1596.
20. Rouillon, C., Athukoralage, J. S., Graham, S., Grüschow, S. and White, M. F. (2019) Investigation of the cyclic oligoadenylate signalling pathway of type III CRISPR systems. Methods Enzymol, 616, 191-218.
21. Gibson, D. G., Young, L., Chuang, R. Y., Venter, J. C., Hutchison, C. A., 3rd and Smith, H. O. (2009) Enzymatic assembly of DNA molecules up to several hundred kilobases. Nat Methods, 6, 343-345.
22. Grüschow, S., Athukoralage, J. S., Graham, S., Hoogeboom, T. and White, M. F. (2019) Cyclic oligoadenylate signalling mediates Mycobacterium tuberculosis CRISPR defence. Nucl. Acids Res., 47, 9259-9270.
23. Elmore, J. R., Sheppard, N. F., Ramia, N., Deighan, T., Li, H., Terns, R. M. and Terns, M. P. (2016) Bipartite recognition of target RNAs activates DNA cleavage by the Type III-B CRISPR-Cas system. Genes Dev, 30, 447-459.
24. Estrella, M. A., Kuo, F. T. and Bailey, S. (2016) RNA-activated DNA cleavage by the Type III-B CRISPR-Cas effector complex. Genes Dev, 30, 460-470.
25. Kazlauskiene, M., Tamulaitis, G., Kostiuk, G., Venclovas, C. and Siksnys, V. (2016) Spatiotemporal Control of Type III-A CRISPR-Cas Immunity: Coupling DNA Degradation with the Target RNA Recognition. Mol. Cell, 62, 295-306.
26 Liu, T. Y., Iavarone, A. T. and Doudna, J. A. (2017) RNA and DNA Targeting by a Reconstituted Thermus thermophilus Type III-A CRISPR-Cas System. PLOS One, 12, e0170552.
27. Park, K. H., An, Y., Jung, T. Y., Baek, I. Y., Noh, H., Ahn, W. C., Hebert, H., Song, J. J., Kim, J. H., Oh, B. H. et al. (2017) RNA activation-independent DNA targeting of the Type III CRISPR-Cas system by a Csm complex. EMBO Rep, 18, 826-840.
28. Johnson, K., Learn, B. A., Estrella, M. A. and Bailey, S. (2019)-Target sequence requirements of a type III-B CRISPR-Cas immune system. J. Biol. Chem., 294, 10290-10299.
29. Marraffini, L. A. and Sontheimer, E. J. (2010) Self versus non-self discrimination during CRISPR RNA-directed immunity. Nature, 463, 568-571.
30. Jia, N., Mo, C. Y., Wang, C., Eng, E. T., Marraffini, L. A. and Patel, D. J. (2019) Type III-A CRISPR-Cas Csm Complexes: Assembly, Periodic RNA Cleavage, DNase Activity Regulation, and Autoimmunity. Mol. Cell, 73, 264-277 e265.
31. You, L., Ma, J., Wang, J., Artamonova, D., Wang, M., Liu, L., Xiang, H., Severinov, K., Zhang, X. and Wang, Y. (2019) Structure Studies of the CRISPR-Csm Complex Reveal Mechanism of Co-transcriptional Interference. Cell, 176, 239-253 e216.
32. Pyenson, N. C., Gayvert, K., Varble, A., Elemento, O. and Marraffini, L. A. (2017) Broad Targeting Specificity during Bacterial Type III CRISPR-Cas Immunity Constrains Viral Escape. Cell Host Microbe, 22, 343-353 e343.
33. Manica, A., Zebec, Z., Steinkellner, J. and Schleper, C. (2013) Unexpectedly broad target recognition of the CRISPR-mediated virus defence system in the archaeon Sulfolobus solfataricus. Nucl. Acids Res., 41, 10509-10517.
34. Manica, A., Zebec, Z., Teichmann, D. and Schleper, C. (2011) In vivo activity of CRISPR-mediated virus defence in a hyperthermophilic archaeon. Molecular Microbiology, 80, 481-491.
35. Goldberg, G. W., McMillan, E. A., Varble, A., Modell, J. W., Samai, P., Jiang, W. and Marraffini, L. A. (2018) Incomplete prophage tolerance by type III-A CRISPR-Cas systems reduces the fitness of lysogenic hosts. Nature communications, 9, 61.
36. Mulepati, S., Heroux, A. and Bailey, S. (2014) Crystal structure of a CRISPR RNA-guided surveillance complex bound to a ssDNA target. Science, 345, 1479-1484.
37. Taylor, D. W., Zhu, Y., Staals, R. H., Kornfeld, J. E., Shinkai, A., van der Oost, J., Nogales, E. and Doudna, J. A. (2015) Structural biology. Structures of the CRISPR-Cmr complex reveal mode of RNA target positioning. Science, 348, 581-585.
38. Welch, S. R., Davies, K. A., Buczkowski, H., Hettiarachchi, N., Green, N., Arnold, U., Jones, M., Hannah, M. J., Evans, R., Burton, C. et al. (2020) Analysis of Inactivation of SARS-COV-2 by Specimen Transport Media, Nucleic Acid Extraction Reagents, Detergents, and Fixatives. J Clin Microbiol, 58.
39. Lima, A., Healer, V., Vendrone, E. and Silbert, S. (2020) Validation of a modified CDC assay and performance comparison with the NeuMoDx and DiaSorin(R) automated assays for rapid detection of SARS-COV-2 in respiratory specimens. J Clin Virol, 133, 104688.
40. Staals, R. H., Zhu, Y., Taylor, D. W., Kornfeld, J. E., Sharma, K., Barendregt, A., Koehorst, J. J., Vlot, M., Neupane, N., Varossieau, K. et al. (2014) RNA targeting by the type III-A CRISPR-Cas Csm complex of Thermus thermophilus. Mol. Cell, 56, 518-530.
41. Tamulaitis, G., Kazlauskiene, M., Manakova, E., Venclovas, C., Nwokeoji, A. O., Dickman, M. J., Horvath, P. and Siksnys, V. (2014) Programmable RNA shredding by the type III-A CRISPR-Cas system of Streptococcus thermophilus. Mol. Cell, 56, 506-517.
42. Hale, C. R., Cocozaki, A., Li, H., Terns, R. M. and Terns, M. P. (2014) Target RNA capture and cleavage by the Cmr type III-B CRISPR-Cas effector complex. Genes & Development, 28, 2432-2443.
43. Nasef, M., Muffly, M. C., Beckman, A. B., Rowe, S. J., Walker, F. C., Hatoum-Aslan, A. and Dunkle, J. A. (2019) Regulation of cyclic oligoadenylate synthesis by the S. epidermidis Cas10-Csm complex. RNA, 25, 948-962.
44. Samai, P., Pyenson, N., Jiang, W., Goldberg, G. W., Hatoum-Aslan, A. and Marraffini, L. A. (2015) Co-transcriptional DNA and RNA Cleavage during Type III CRISPR-Cas Immunity. Cell, 161, 1164-1174.
45. Grüschow, S., Athukoralage, J. S., Graham, S., Hoogeboom, T. and White, M. F. (2019) Cyclic oligoadenylate signalling mediates Mycobacterium tuberculosis CRISPR defence. Nucl. Acids Res., 47, 9259-9270.

1.-25. (canceled)

26. A system for the detection of a target nucleic acid, said system comprising:
a CRISPR associated protein 10 (Cas10);
a molecule or processed form thereof capable of binding the target nucleic acid;
a nuclease; and
a reporter system.

27. The system of claim 26, wherein the target nucleic acid comprises RNA or viral RNA.

28. The system of claim 26, wherein the system further comprises ATP.

29. The system of claim 26, wherein the molecule or processed form thereof capable of binding the target nucleic acid comprises nucleic acid complementary to at least part of the sequences of the target nucleic acid.

30. The system of claim 26, wherein the molecule or processed form thereof capable of binding the target nucleic acid comprises a guide RNA comprising a sequence which is complementary to a sequence of, or present within the target nucleic acid and/or binds to a target nucleic acid or to a target sequence within the target nucleic acid.

31. The system of claim 30, wherein the system comprises two or more guide RNA, wherein each guide RNA binds the same or a different target nucleic acid.

32. The system of claim 26, wherein the CRISPR Cas10 component is derived from a type III CRISPR complex.

33. The system of any preceding claim, wherein the Cas10 component is from Vibrio metoecus.

34. The system of claim 26, wherein the system further comprises one or more additional CRISPR associated proteins.

35. The system of claim 26, wherein the system comprises
one or more (for example two, three or more) Cmr1 protein(s); and/or
one or more (for example two, three or more) Cmr3 protein(s); and/or
one or more (for example two, three, four or more) Cmr4 protein(s);
one or more (for example two, three, four or more) Cmr5 protein(s); and/or
one or more (for example two, three or more) Cmr6 protein(s).

36. The system of claim 26, wherein the system further comprises Cas6.

37. The system of claim 26, wherein the reporter system comprises a nucleic acid.

38. The system of claim 26, wherein the reporter nucleic system comprises a double-stranded DNA (dsDNA); an optically detectable label; and/or a quenched optically detectable label.

39. The system of claim 26, wherein the nuclease is activated by cyclic tri-adenylate (cA3).

40. The system of claim 26, wherein the nuclease does not have a CARF domain.

41. The system of claim 26, wherein the nuclease comprises a NucC nuclease.

42. The system of claim 26, wherein the nuclease comprises NucC from Vibrio metoecus.

43. The system of claim 26, wherein the nuclease comprises the sequence of SEQ ID NO: 3:
(a NucC sequence from Vibro metoecus (WP_
000046098.1))
SEQ ID NO: 3
MAQDWQLSELLENLHADVQHKLTTVRKSFKHSVVKGDGAENVWVDLENQ
YLPERYRASRAFVVDSENQFSEQIDVVIYDRQYSPFIFHYAEQLIIPAE
SVYAVFEVKQTLNKQHIDAARKKVASVRALHRTSLPIPHAGGVHSPREL
IGIIGGLLTLENELKIPDTLMGHLDHDKADKGMLNIGCAADDCFFYYDN
DHQRMQVMQHKKATTAFLFELLSQLQKCGTVPMIDIHAYGKWLTPRISE

44. A system for the detection of a RNA in a sample, said system comprising:
a Vibrio metoecus CRISPR associated protein 10 (Cas10);
a molecule or processed form thereof capable of binding the target nucleic acid;
a NucC nuclease;
a double stranded DNA reporter system comprising a quenched fluorescent label; and optionally
ATP; and/or
Cas6 and/or
one or more Cmr1(Cas7) protein(s); and/or
one or more Cmr3 (Cas5) protein(s); and/or
one or more Cmr4 (Cas7) protein(s); and/or
one or more Cmr5 (Cas11) protein(s); and/or
one or more Cmr6 (Cas7) protein(s).

45. A system of claim 44, wherein the system comprises a guide RNA for binding to the target RNA.

46. A method of detecting a target nucleic acid in a sample, said method comprising contacting a sample with the system of claim 26, wherein detection of a signal from the reporter system, indicates that the sample contains the target nucleic acid.

47. The method of claim 46, wherein the method is for the detection of a RNA or a viral RNA in a sample.

48. The method of claim 46, wherein the method is for the detection of SARS-COV-2 RNA in a sample.

49. A nucleic acid or an expression vector comprising one or more of the following nucleic acid sequences:
a nucleic acid encoding a Cas10 protein; and/or
a nucleic acid encoding a Vme Cas10; and/or
a nucleic acid encoding a molecule which is capable of binding the target nucleic acid; or a molecule which can be processed (by for example Cas6) into a molecule which binds the target nucleic acid and/or
a nucleic acid encoding a nuclease; and/or
a nucleic acid encoding a NucC nuclease, for example a VmeNucC; and/or
a nucleic acid encoding a reporter system.

50. The nucleic acid or expression vector of claim 49, further comprising:
a nucleic acid encoding a Cmr1 protein; and/or
a nucleic acid encoding a Cmr2 protein; and/or
a nucleic acid encoding a Cmr3 protein; and/or
a nucleic acid encoding a Cmr4 protein; and/or
a nucleic acid encoding a Cmr5 protein; and/or
a nucleic acid encoding a Cmr6 protein.


SEQUENCE LISTING (33 sequences)

SEQ ID NO: 1 - RNA, 32 nt, SARS-CoV-2
aaggcguuccaauuaacaccaauagcagucca

SEQ ID NO: 2 - RNA, 32 nt, SARS-CoV-2
gaugguauuucuacuaccuaggaacugggcca

SEQ ID NO: 3 - protein, 245 aa, Vibro metoecus
MAQDWQLSELLENLHADVQHKLTTVRKSFKHSVVKGDGAENVWVDLFNQYLPERYRASRAFVVDSENQFSEQIDVVIYDRQYSPFIFHYAEQLIIPAESVYAVFEVKQTLNKQHIDAARKKVASVRALHRTSLPIPHAGGVHSPRELIGIIGGLLTLENELKIPDTLMGHLDHDKADKGMLNIGCAADDCFFYYDNDHQRMQVMQHKKATTAFLFELLSQLQKCGTVPMIDIHAYGKWLTPRISE

SEQ ID NO: 4 - protein, 242 aa, Gynuella sunshinyii
MSDWKLSQLLESLHGDIQHRLKTVRQTIEHPTMKGDGSENVWIGLLNNYLPERYRSSRAFVVDSNGEFSEQMDVVIYDRQYSPLVFHYEEQLIIPAESVYAVFEVKQTFDKGHIDAAHKKVASVRKLYRTSMDIVHAGGISKSRTPFSIIGGILALECDLKELETTLKGYLMGADRNDESKWLTSGCAANRCFFYHDKEHHDIKISQHPKATTAFLFQLLSQLQSCGTVPMLDIHAYGKWLE

SEQ ID NO: 5 - protein, 239 aa, Eubacterium sp.
MDIKSLFNTKQTILESKLSVLLEHPVTKGEHCESAWIDFFRSFLPNKYAVDKGFVFDSKGNVSDQIDIIIYDALYAPLIFGTDAGEKFITAESVYAVFESKPKINKKTLAYANNKIESVNVLERSARGVINAGKYYPPRNLTKIIGGILSIDSINIDKIEKHLNLFKYIDLGCAIKNHSFLVNRESKLSLLHASGEETVLAFFYMILDELYKLGTVAGIDIRNYANANLDNFKFDLDKK

SEQ ID NO: 6 - protein, 250 aa, Serratia sp.
MTNQAKKLSRINGREFLKQSFNLQQQLLASQLNLSRTITHDGTMGEVNESYFLSIIRQYLPERYSVDRGVVVDSEGQTSDQIDAVIFDRHYTPTLLDQQGHRFIPAEAVYAVLEVKPTINKTYLEYAADKAASVRKLYRTSTVIKNIYGTAKPVEHFPIVAGIVAIDVEWQDGLGKAFTENLQAVSSDENRKLDCGLAVSGACFDSYDEEIKIRSGENALIFFLFRLLGKLQSLGTVPAIDWRVYIDSLE

SEQ ID NO: 7 - protein, 250 aa, Pectobacterium carotovorum subsp. Carotovorum
MTSQAKELSNINGREFLKNSFDLQQKLLASQLNLSRTITHNGTMGDVNESYFISIIRQYLPERYSVDRGIVVDSKGQTSDQIDVVIFDRHYTPTLLDQQGHRFIPAEAVYAVLEVKPTINKTYLDYAADKAASVRKLYRTSTVIKNIYGKAKPVEHFPIVAGIVAIDVEWQDGLGKAFTANLQAISSDGNRKLDCGLAVSGACFDSYDEEIKTKSGENALIFFLFRLLGKLQSLGTVPAIDWRVYIDSLE

SEQ ID NO: 8 - protein, 307 aa, Dickeya zeae
MTNQAKELSKKDGKKYLKNAFKLQQEVLKTQLLQSRAAITHEGVKGDVNEGYFLDIIRQYLPERYSVDRGIVVNSAGRTSDQIDVIIFDRHYTPTLLNQQGHRLIPAEAVYAVMEVKPIIDASNLDYAADKAASVRSLDRTNMTFRHSGGVGRGRLFNIITGIIAIDVDWRDGFNSDAFKKKLQSINQSEDNGHKNLDCGLALSGGCFDFFSKEDEKIKKEEERKKKEEEEKRNRDPQSQSEINVKEIFKTIENTQMENLTIRNKEEGALAYFLFRLLRELQWLGTAPAIDWNEYVNMLDHDNEPST

SEQ ID NO: 9 - protein, 256 aa, Marinomonas mediterranea
MNNAKTKSDTDGEQYLKDSFQREQELLESKLLFANQNITHNGERGEVNEKHFIEYLRSHLPKRYSVDSAIVVDSNGKTSNQIDIVVYDNQYTPTLFAQQDFCYVPAEAVYAVIEVKPEVNRDYIEYAQKQAYSVEALERTSIEITHAGGVYKPKPLFKIITGLIALKTGWQEGLASNSFKDAIQMPYTNQTRLDFVTSLQGGHFDCFEKGNEHYSQQNHLTFFLFRLLSQLQSLGTVPAVDWNKYANVLTEQSKPD

SEQ ID NO: 10 - protein, 241 aa, Thioflavicoccus mobilis
MNSLSRLFASLHEDLERQLSITRESLRHPSAKGDESESIWLALLQNYLPTRYRAERAQVVDSEGRFSDQIDVVVFDRQYSPFIFQLGNQRFVPAESVYAVFEAKQSIGPREITYAKKKICSVRALFRTSLPIPHAGGEYEPKPLTPIIGGVLSLESNWNPPLGEPLRKALETNDPMSRIDIGCIASRGLFHSRDGAQTFEFHVGCKTAARFLLRLISELQRIGTVQMIDIMAYERWLVDDR

SEQ ID NO: 11 - protein, 302 aa, Paenibacillus popilliae
MSEPILDHKIIQGIASNYRRLEQAIVDQLRMSSHHHVTSGGFREEMWKQLFEQIIPKKYSVARSVFIIDSEGKVSKEVDLAIFDEQYTPYIFRYGQMKYIPIEAVAVAIQCKSSLSDYDGIKDWTDSIAKLNTSMKSITRIQSGVVCGEFDFMTENGEFILREGQRPTAQTATRPLIILCHLDEGFTSKNSLVEYFDFIICPTPTGWLRAFVNKEGHTLEAWYEALNHTNEKYKNVRSPFGKSSAGNCTLKNYKVHAPDEPENDISLLTLTFQLNQLLMLINNPILFPHQAYVEMFNRSLKE

SEQ ID NO: 12 - DNA, 769 bp, Artificial Sequence
  Synthetic DNA
gcgcccatggcacatatggcccaggactggcaactttctgagcttttggaaaacttacacgccgatgtgcaacataaactgaccaccgtccgcaaatcttttaagcattcggtagttaaaggtgatggtgctgagaacgtctgggttgatttgtttaaccaatacttgccagagcgctaccgcgccagccgcgccttcgtggttgactcagaaaatcaattttcggagcaaattgacgtagtcatttacgaccgtcagtacagcccctttatctttcattacgcggaacagttaatcattccggcggagtccgtatatgccgtatttgaggtcaaacaaacgctgaataagcagcatattgacgctgcacgtaagaaagtggcgtcggtgcgtgcacttcaccgtacgagtttgcccatcccccatgctggcggagtccactccccacgcgaacttattggtattattggaggtttacttaccctggaaaacgaactgaaaattccagacacactgatgggacatttggatcatgacaaagccgataaggggatgttgaacattggttgtgcagccgatgattgtttcttctactacgacaacgaccatcaacgtatgcaggtcatgcaacacaaaaaggcgacgacagcattcctgttcgagctgttgagtcagttgcaaaaatgtgggacagtccctatgattgacatccacgcctacggcaagtggttgacccctcgtatttctgagtgactcgaggtcgaccgcg

SEQ ID NO: 13 - DNA, 569 bp, Artificial Sequence
  Synthetic DNA
gcgccatatggactcatacatcgatattcatttgcgccctgacgccgagatgaatgaagctgaacttgggagcaaagtatatactaagtttcataaagcattggtgaaactgaatacgaaccaaatcgcgatctcgtttccaggggctaaccttaagctgggacaattattccgcgttcatggccccgtttcgcttttgaatgacttgcaaggtctgtgctggctgggtccattatctggctattgtcaaatctcagaggtgctttccgtacctgagcaggtacaatatcgcgtaatctctgcaaaacgccgtaatctttcggcggcaaaattgcgtcgcctgattgcacgcgggtccattaacaaggagggggaacaacgttacaaaaaaaaaatgttaaaccaatcgatcaagggaccttatctggatttgctgtcctctagcacaggccaaaaatatcgtaaattctttgaatttggtgaaatccaagatgtgcccgttctgggaaatttcgatacctatggcttatcactgaaggctacagtcccctggttttgactcgagcgcg

SEQ ID NO: 14 - DNA, 216 bp, Artificial Sequence
  Synthetic DNA
cgcgccatggtaaaaatacaatttttaccctaactgactgttgtaacttacttttatagatttattctatagttcactgccgcacaggcagcttagaaagtgtcttcgtaccttgaagaccagttcactgccgcacaggcagcttagaaaaatctttatatatcttatggttgcagatctaaaaagttgggattatataaatgacagtcgacgcgc

SEQ ID NO: 15 - DNA, 1203 bp, Artificial Sequence
  Synthetic DNA
atgcgccgtcagaataacaccatcgatttacagggacttaagaaagaacttcttaacagcaacaaagtcaacgataagtgggagagttattcctgcacgctggtcacgccaatgtacggaggtggagtaaaggcgggagaggtggacaaagatatgcctattcgcgcatccgcaatccgtggccagttacgcttttggtggcgtattgcgtgtgggtccaaggcaccagaagtaatgcgtgaaaatgaggaagctatctgggggggcattggtgacaaagctgcaattgcctctcaggttcagatccgcgtcatctcgaaaaatgtaatcatgtccaacttagtcagctcgaaaaaattggcaggttctggggtcaagtacgctctgggagccgcagacgaggcgtattgtttacctagtggatacaactttgtgttggaaatccgctataaggacgatattacatccgatcagatcaagcaggtgaaagaatcattgcgctggtggagctcattcggcggggtaggggccaaaacacgccgtggcttcggggctgttgttgttgatagcattaaaactatcgaagcaacggaggtggaaacgatcggtggtaaaatcgctttaactgagcagtcctcagactcggcgcaagatatgtggaaaaaggccaccgagcttttatataagtttcgtcaaggccgcgagttggggcgcaatgaaggtcaagggaaccgtcctggccgttcacgctggcccgagccggatcaattacgccgtatgtcaaacaagcataaaacaaatcacgagccagaacataaggcgggaaatgtgtttccacgcgctgcatttggtatgccaatcatttttgattttaacgaccgtagtcgtacagaaccatctactatgacacttcttcccaaagatgcccaacgtatggctagccctcttattatccgtccctacaaaaatggagatcaatggcaagcagcagcattgttgcttccaaattggcagacggcattaaacgagcccttggagttgagtcctacaccaaacaacgggacacctaatcattggcccacctcggagaatgagcgtactcgtttagcagaaattattcgccctatggtcggaaagaatggccaattgcgtgcgaacgatccactgtcagcgttccttgacttttttgagaatggccaataa

SEQ ID NO: 16 - DNA, 1842 bp, Artificial Sequence
  Synthetic DNA
atgacagactacctggttgccatttctatcggtccagtacagtcgctgatcgaagcaggccgccgcgcgcaagatctgtggtgcggatcatggttactgagcgaagttgcccgtgccgttgcgttgaagctgcaccaaactcagaactcgtgcctgatcttccccaacccagaacacccccaagcggaattacttccgcaatccagctccgacgaaccgcgcgcgaacatcgcgaatgttatccgcgctgttatctctgccgattcgaaagaggaacttcaaaataagttagaaaaagcaaaaggagcggccttcgatcgccttttcggtatttttacggaagtgttatcgcaaaacgaacttcagaatcttggcatcgaccgcgcccgctggcagcagcagcaaaatgatgtccttgagatctttagtgcttgggtttctttagataaccacgaatacaacgctgcgagcattcgccttggtaagttgttgcatgctcgcaagttatcgcgcaatttcgagcctatgaaggactgcatggctgcgctgcataaaagtactctggacgggtccaacaacaccgttaccaataaattaactgcgcgtgatgcggctaaaaagctgcacatttcagatcaagaacttcagcaaaaacgccgttttttaggtttgacgaacgaagaggaattagacgcattgggagtagtgaaacgtcgcgccggcaatttggaacagttcacgccgttttcgcgcattgtagcacatggatggttgaattccctgtctgatgaacaacgtgcaggcttgaaggtggcatatcaacccttccttgatagcggacacgtaacgcaagtaaaaggaaacgatggcatctacgctgattttccttttgatggtgagtatctgttcttgagccgtctggcgcaagcagacattgatcatgatctgaaagataaattacagaagcaattagcagcaatcaagtcatcacctgttccctatggtgttctgcttaaggcggatggagatcgtatgggggatttacttagcaaagcggaaggcaagcagcagtcgaaggcaatctcgaaggccttacacgaattcgctacatccgttcgtaaaaccttgcaggatcatgggggtcatgcgatttatgcgggcggggacgatgttcttgcttttgtgccccttgctcaggcaatgacctgcgcgaaacaattggcggacgatttcaaggaaaaaatgaaggtgattgcggccgaattaaaacttagtgaagcattatacccgacattaagcgtcggtctggctatcggtcattttgtgcaacccatgcgccaacttcgtgcacgtgccatcgctgcggaaaaacacgccaaaggcaacaaagagcataaaccacgcaatgcgttggccattcaccttggtattcgctctggccatgagatcacctggcgctgtcgctgggatgacgatgaaaccttaaacgcactgactgacttcacgcatgcatttgcccagggctggatgcccacccgtattatgcaagaggttcgcgagatggcagtgcacttaaaatggacaacgggacaaaaagagctggcaggaattcgtgagagcgagttagaacgtatgcttgcacgcgcggagttcaacgtgagtgtagaacagtcaggtaagactcctgagcaactgaaggaaagtaagaaagcggctttagataagttaaaatcacagttacgtgcacaagcatccggttcgcttgatgaattagcgaacctgttgatcttggcccgttggttaagcgccaaaactacagcggatatcggtggagaggagtaa

SEQ ID NO: 17 - DNA, 1161 bp, Artificial Sequence
  Synthetic DNA
atgttatattatcttattgaacctaaagatcccttaattatccgctccggccgtccattcgaagagatctcagatgcgcaggcggcgcgtttcccacctccaagtactgtagcgggagcgttacgcaacatccatgctcgtagtacaggtaaaactttggataacaagcttttgaaacttgacaacgaattgctgaaacttgccgttactggcccgctggccgttaagcgtcccattaacggtgacgccccctcggaagaacacattcttgtaccgaagcccgcagatgtgcagtacttctacgatcagcagacgcagttgacccatcttgtgcgttcgaagccaatggcgttcgcagagggtgaaggttgcgaccttccgaacggactgttaccgttattcgccgagaacgcgcctgatgggaagcccgtttctgggcccaactggtggtccttcaatgacttagctgcgtggcgcaaaggccagtcagtatcattcgagcgtatttgtcagaacggctggatgcctgcggaaccagatatccgcacacacattgctattaacaaccattcccgtaatgtagagagtggtaagcttttccagacgacagggttgtccatgtggcagcaacgtgcggatcatcaaccgttcccagacgcatgtgttagcatcttggctggcattgacggtgatattacccttccgttgattaatctgggtggcgagcgccgtcttgctgaagtcgaagcttgtactttatggccttctctgcccagcgacttggcccaatccatcacgaaggccaaaggcttcactttaacgtttttgaccccagtgctttttaattctggatggttaccgagttggttaaacgatgagttaatcgggacacctccctgctgtcagagtctgaccgttaaacttcgcgcagccgcacttgaacgctggatccctcaatcaggttgggacttggtaaataatacaccccgtgcagctcaaaagatgattccggcaggtgcgacctattggtttgagatcgaaggagaggccaccgacgaggatattcgctcactttggttagcccatttttgtgacgacccccagagcaacctgaacgggtttggtctggctttgcccgtggcgtatcagtttactctgtaa

SEQ ID NO: 18 - DNA, 879 bp, Artificial Sequence
  Synthetic DNA
atgagttttcatgtttaccatctgttttcccaaacgatcttacactgcggatcgggccagagtgtcggtattgttgaccaacccatcgcgcgcgagcgcgcatctaatttgcctatcgttcctggatcgaccgtccgtggtgtgcttaaagccttcatcagtcacacagaacagactgaggtaaaaccgacccttagtcagagcttgttcggatatgacaagcaagacggtgaaccaagttttgcaggcgctttgagtattacggacgctcatttgcttcttttacccgtccgcacggtatacggtatccttgcctatgctacatgtccgtttatcttgcaacgctataaaaaagaccgcaaacttgatcttatggtgccagctcctgctgatgaacaagcacttcacccgaaaggaaacccaaatcaccaagataatcttatggtcttagaagatttagatttgaaggtgcaagaatgcgaccatactcaacaatgggctgagtacattgctcaaacactgtacgcaaaagactcagcttatttcagcgacatgagcaagcgtatcattgtcttacccgatacagtcttttccttcctggctgaaacagccacggagattcgcacgcgcatccgtattaaccaggagacaggagttgtagataatggcgcactttggacagaagaaagcttgccggctgaatccgtgttgtggggagtatacagtgtcgacgcatcccgcttaaacgacaagaacggagataagcaacttatgtttaatgacattatcaacaataagcccctgttgcaaattggcggcaacatggggacgggtaacgggttagttcagttcattgcgcagtctgcccgtggcgagtaa

SEQ ID NO: 19 - DNA, 423 bp, Artificial Sequence
  Synthetic DNA
atgcaaccacgctcgcagattgttgcgacggcagcattcaagcaaatcaattcacgtaagaacaagtccattgaggagaacaagaaatatgctaccttggcgcataaattgcctaccatgatcttacaaaacgggcttgctcaggccaccgggttcttgttaagtaaaagcgaagaacatcataaagctctgttagaagacttggtgttggtgtttaaacaagtggacgcgaagctggctaacattgcaaacgccgaagccttacacgatgcgattatccaaagtgatttgccacaaattatgcgcatgactcgtgaggcccttgaaatcgcgggttggttacgccgttacgttcaaggtgtgttgaaaattgacgcgacgggggaaccgatggataaaaagcccaatgagaaaggagcatga

SEQ ID NO: 20 - DNA, 1125 bp, Artificial Sequence
  Synthetic DNA
atggttcaattagttcgtgacaaattaaaagaagcgttcgatcattccgacagtatcaacccctcgctgttgctgcaaaaaggattgttagagaagaatagtgatgccaagtcagacaacaataagacggggcacttgaacaagattgtaaaactgcctgcctcgcctgaatataaaaatggctttaatcgctggttcgacttaacgcttgatgaaaatcgcttttcccagaccgctatgacgttggaaaatcgtcttttaattggcttgaccggtcagggggccttggaaaccggttgctcgctttcacgtaattatggtatgccgtatattccgggttcctcagttaagggggcggtccgcgcgtgggctaatcagcatcttgctggacactccgacgagctggagcagttgtttgggacggccgattccgaacaaccctatcgcgtgtctggacttgtcacatttcacgacgcttggtggattcctgatcccgcaaaaaaggagcacaagcctttcgttctggatgtagtgacaacacatcaccaggcatactacaatgggacccaggcggagcccagtgataaagatagccccattccaaaccaccttctggccgtacaaggatcttttttattcgtattagaaggggagtcagctgctattgagttgtgtcagactctgttagaaaaagctctggccaacaacggcatcggtgcaaaaaccgccgccgggtacggctacatgaaggttgatccagcgttgatgcaacgcctgctggacgagtatgaaaagcgtctgtcaccagaagaacgcgaacaccgtgaggctgaagctcaacgccgtaatgaacaacaactggaagcagccgcaaaagcagaacaagcgaagcccccagttcagatcattgcggagttacgcgctggctatctgaaacatcgcgataatgcggcttaccagttaaaggtagataattgggtcgacacagcaatcaaaacttggacgacgcaggatcgtacgttattagcggagtgcttaaagcaggtgggctacgagccgagtaataagaagaacccaaatcacttaattcgcaaaacccgtttgcaacaattgaagggcaaatga

SEQ ID NO: 21 - DNA, 28 bp, Artificial Sequence
  Synthetic DNA
gttcactgccgcacaggcagcttagaaa

SEQ ID NO: 22 - DNA, 36 bp, Artificial Sequence
  Synthetic DNA
gaaagaattcgagctcggtacccggggatcctctag

SEQ ID NO: 23 - DNA, 36 bp, Artificial Sequence
  Synthetic DNA
gaacctagaggatccccgggtaccgagctcgaattc

SEQ ID NO: 24 - RNA, 44 nt, Artificial Sequence
  Synthetic RNA
gacucuagaggauccccggguaccgagcucgaauucaaagauuc

SEQ ID NO: 25 - RNA, 48 nt, Artificial Sequence
  Synthetic RNA
aggguauuauuuguuuguuucuucuaaacuauaagcuaguucuggaga

SEQ ID NO: 26 - DNA, 30 bp, Artificial Sequence
  Synthetic DNA
agtgttacattatccaccatggcgagcttt

SEQ ID NO: 27 - DNA, 30 bp, Artificial Sequence
  Synthetic DNA
aaagctcgccatggtggataatgtaacact

SEQ ID NO: 28 - DNA, 328 bp, Artificial Sequence
  Synthetic DNA
gttcactgccgcacaggcagcttagaaaacgctgaagcgctgggggcaaattgtgcaattgttcactgccgcacaggcagcttagaaaatcgcgccccactgcgttctccattctggttagttcactgccgcacaggcagcttagaaagcaaagcaagagcagcatcaccgccattgccagttcactgccgcacaggcagcttagaaagatggcacctgtgtaggtcaaccacgttcccggttcactgccgcacaggcagcttagaaatgttacattgtatgctttagtggcagtacgttgttcactgccgcacaggcagcttagaaa

SEQ ID NO: 29 - RNA, 44 nt, Artificial Sequence
  Synthetic RNA
gcaguaaccagaauggagaacgcaguggggcgcgaucaaaacaa

SEQ ID NO: 30 - RNA, 44 nt, Artificial Sequence
  Synthetic RNA
uggcuggcaauggcggugaugcugcucuugcuuugcugcugcuu

SEQ ID NO: 31 - RNA, 44 nt, Artificial Sequence
  Synthetic RNA
caaaaacguacugccacuaaagcauacaauguaacacaagcuuu

SEQ ID NO: 32 - RNA, 44 nt, Artificial Sequence
  Synthetic RNA
cgcaaauugcacaauuugcccccagcgcuucagcguucuucgga

SEQ ID NO: 33 - RNA, 44 nt, Artificial Sequence
  Synthetic RNA
ccuucgggaacgugguugaccuacacaggugccaucaaauugga